(19) United States
(12) Patent Application Publication
(10) Pub. No.: US 2020/0317784 A1
(43) Pub. Date: Oct. 8, 2020

(54) METHODS AND COMPOSITIONS FOR TREATING CANCER BY MODIFYING MULTIPLE ARMS OF THE IMMUNE SYSTEM

(71) Applicants: BioXcel Therapeutics, Inc., New Haven, CT (US); Nektar Therapeutics, Inc., San Francisco, CA (US)

(72) Inventors: Krishnan NANDABALAN, New Haven, CT (US); Vimal D. MEHTA, New Haven, CT (US); Luca RASTELLI, New Haven, CT (US); John MACDOUGALL, New Haven, CT (US); Jonathan ZALEVSKY, San Francisco, CA (US); Deborah H. CHARYCH, San Francisco, CA (US)

(21) Appl. No.: 16/763,378

(22) PCT Filed: Nov. 13, 2018
(86) PCT No.: PCT/US18/60699

§ 371 Date: May 12, 2020

Related U.S. Application Data
(60) Provisional application No. 62/584,999, filed on Nov. 13, 2017.
(60) Provisional application No. 62/629,473, filed on Feb. 12, 2018.
(60) Provisional application No. 62/679,576, filed on Jun. 1, 2018.
(60) Provisional application No. 62/712,457, filed on Jul. 31, 2018.

(51) Int. Cl. C07K 16/28; A61K 38/20; A61K 31/69

(52) U.S. Cl. CPC C07K 16/2818; A61K 45/06; A61K 31/69; A61K 38/2013

(57) ABSTRACT

Provided herein are combination methods and compositions for cancer therapies. The combinations modify multiple arms of the immune system, including an innate immunity modifier, an immune checkpoint inhibitor and a T-cell stimulator, to treat cancer.

### CROSS REFERENCE TO RELATED APPLICATIONS

This application claims the benefit of priority under 35 U.S.C. 119(e) to U.S. Provisional Patent Application No. 62/584,999, filed on Nov. 13, 2017; U.S. Provisional Patent Application No. 62/629,473, filed on Feb. 12, 2018; U.S. Provisional Patent Application No. 62/679,576, filed on Jun. 1, 2018, and U.S. Provisional Patent Application No. 62/712,457, filed on Jul. 31, 2018, the disclosures each of which are incorporated herein by reference in their entireties.

### DESCRIPTION OF THE TEXT FILE SUBMITTED ELECTRONICALLY

The contents of the text file submitted electronically herewith are incorporated herein by reference in their entirety: A computer readable format copy of the Sequence Listing (filename: BXTI-001_01WO_SeqList_ST25.txt, date recorded: Nov. 12, 2018; file size: 4 kilobytes).

### FIELD

The present disclosure relates to, among other things, a combination therapy comprising an innate immunity modifier, an immune checkpoint inhibitor and a T-cell stimulator for treating a subject having cancer, as well as related compositions and methods.

### BACKGROUND

The National Cancer Institute has estimated that in the United States alone, 1 in 3 people will develop cancer during their lifetime. Moreover, approximately 50% to 60% of individuals contracting cancer will eventually succumb to the disease. Despite advances in cancer therapy, existing therapeutic modalities still fail to adequately control or cure certain cancers. Often those patients who initially respond to anti-tumor treatment later relapse, indicating, for example, that the tumor has mutated in a manner that eliminates the therapeutic benefit of the treatment modality employed. The use of therapeutics to generate an immune response against cancer cells which are intrinsically recognized as “foreign” by the immune system due to the production of abnormal variants of proteins as a consequence of mutations has recently shown promise in cancer treatment regimens.

Immune checkpoint inhibitors have been used successfully to treat cancer patients, in particular, patients with non-small cell lung cancer (NSCLC), metastatic melanoma or Hodgkin lymphoma. Checkpoint inhibitors have also shown promise in clinical trials involving patients with other types of cancer (O'reilly A et. al., Expert Rev Anticancer Ther. 2017 July; 17(7):647-655). Unfortunately, the use of immune checkpoint inhibitors suffers from several limitations. For example, only a minority of patients treated with immune checkpoint inhibitors exhibit robust anti-tumor responses, and most responses are partial and temporary. Many patients initially respond to immune checkpoint inhibitor-based therapy, and then relapse due to the emergence of resistant pathways. Such resistant pathways may occur for a number of reasons, although a primary reason may be due to the generation of non-immune permissive micro-environments by the tumor cells (the so-called “non-inflamed”) (Gajewski T F., Semin Oncol. 2015 August; 42(4):663-71; Gide T N et. al Clin Cancer Res. 2018 Mar. 15; 24(6):1260-1270). Reports have indicated that the use of certain immune checkpoint inhibitors has led to deaths associated with their cardiotoxic side effects (Moslehi J J et al., Lancet. 2018 Mar. 10; 391(10124):93; Heinzerling L et al., J Immunother Cancer. 2016 Aug. 16; 4:50). Recently, a combination of the two immune checkpoint inhibitors, ipilimumab and nivolumab, was shown to increase the response rate in melanoma patients from the 11% and 32% seen with the respective monotherapies, to 60% with the combination (Postow M A et al., N Engl J Med. 2015 May 21; 372(21):2006-17).

Despite these advances, there remains a need to identify and provide new and effective anti-cancer treatment regimens. The present disclosure seeks to address this and other needs.

### SUMMARY

The present disclosure provides improved immunotherapeutic modalities for treating cancer. More particularly, therapeutic combinations, compositions and methods that utilize both the adaptive arm of the immune system and an innate arm of the immune system are described, and are shown to be effective to provide a notable anti-tumor effect in illustrative animal models. Thus, the present disclosure provides unique combinations for treating a subject having cancer, wherein the combinations are effective to modify multiple arms of the immune system (as described above) to thereby facilitate enhanced immune system-based attacks on cancerous tumors, and provide robust anti-tumor effects. Without being bound by theory, it is thought that the activation of the T-cell pathway promotes T-cell tumor infiltration, which in combination with inhibition of immune checkpoint inhibitor activity, promotes enhanced general anti-tumor activity. Thus, it has been recognized by the Applicants that by collectively combining these three discrete therapeutic axes into a single treatment regimen, a broad and diverse stimulation of the immune system can be effected to elicit a significant anti-tumor response.

More particularly, in a first aspect, provided herein is a therapeutic method for treating a subject having cancer, the method comprising administering to the subject an innate immune modifier (i.e., an agent that primarily stimulates the innate immune system), an immune checkpoint inhibitor (i.e., an agent that inhibits the immune checkpoint involved in immune escape as harnessed by the cancer-progressing tumor microenvironment), and a T-cell stimulator (i.e., an agent effective to activate the adaptive arm of the immune system primarily composed of effector T-cells).

In some embodiments of the method, an effective amount of each of the innate immune modifier, the immune checkpoint inhibitor and the T-cell stimulator, optionally together with one or more additional anti-cancer agents, such as one or more innate immune modifiers, immune checkpoint inhibitors and/or T-cell stimulators, is administered.

In some particular embodiments of the method, the innate immune modifier is a selective dipeptidyl peptidase inhibitor, the immune checkpoint inhibitor is a PD-1 axis antagonist or a CTLA-4 antagonist, and the T-cell stimulator is an interleukin-2 (IL-2) or is a modified form thereof, such as, for example, a prodrug of an interleukin-2 (e.g., aldesleukin) in which the interleukin-2 is modified by releasable covalent attachment of multiple polyethylene glycol moieties. In yet one or more additional embodiments, the T-cell stimulator is an interleukin-2 receptor beta (IL-2Rβ) selective agonist.

In a second aspect, provided herein is a method of enhancing an immune response in a subject, the method comprising administering an effective amount of a combination of therapeutic agents comprising an innate immune modifier, an immune checkpoint inhibitor and a T-cell stimulator, wherein the subject has been diagnosed with cancer.

In yet a third aspect, provided is a pharmaceutical combination comprising (a) a therapeutically effective amount of an innate immunity modifier, (b) a therapeutically effective amount of an immune checkpoint inhibitor, and (c) a therapeutically effective amount of a T-cell stimulator (also referred to herein as a triple combination), e.g., for treating a patient with cancer.

In yet a forth aspect, the present disclosure provides a pharmaceutical composition comprising: (a) an effective amount of an innate immunity modifier, (b) an effective amount of an immune checkpoint inhibitor, and (c) an effective amount of a T-cell stimulator, together with one or more pharmaceutically acceptable carriers and/or excipients.

In one or more embodiments related to the foregoing methods or use of the combination, the innate immunity modifier, the immune checkpoint inhibitor, and the T-cell stimulator are administered to a subject at the same time (separately or together as part of a single pharmaceutical formulation), sequentially and in any appropriate order, or are administered separately (e.g. intermittently), via the same and/or different routes of administration, each in an immunomodulating amount.

In yet some further embodiments, when administered separately, each of the innate immunity modifier, the immune checkpoint inhibitor, and the T-cell stimulator is comprised in a pharmaceutical composition, e.g., in a form suitable for administration via an appropriate administration route.

In yet some additional embodiments, treatment may comprise a single cycle of therapy, or may comprise multiple (i.e., two or more) cycles of therapy, where multiple cycles of therapy may comprise administration of each of the innate immunity modifier, the immune checkpoint inhibitor, and the T-cell stimulator, or may comprise administration of fewer than each of the initially administered immunomodulating agents.

In some preferred embodiments, the subject is a human subject.

In some additional embodiments, the subject is a human subject that was previously non-responsive to immune checkpoint inhibitor therapy.

In yet some further embodiments, a preferred combination, composition, or method comprises (a) talabostat mesylate, (b) a PD-1 axis antagonist, and (c) an interleukin-2 receptor beta (IL-2Rβ) selective agonist, such as, for example, a PEGylated interleukin-2 (i.e., an interleukin-2 protein conjugated to polyethylene glycol).

Additional agents and/or therapies can be administered or provided in combination with the triple combination therapy described herein. In some embodiments, the one or more additional therapeutic agents comprises a cytotoxin and/or chemotherapeutic agent.

In yet some further embodiments, the cancer is selected from breast cancer, hematopoietic cancers (such as AML and CLL), head and neck cancers, sarcoma, fibrosarcoma, colon cancers, colorectal cancers, pancreatic cancers, skin cancers, and lung cancers. In one or more particular embodiments, the cancer is pancreatic cancer. In yet some further embodiments, the cancer is colorectal cancer. In yet some other embodiments, the cancer is sarcoma. In one or more related embodiments, the cancer is fibrosarcoma. In some additional embodiments, the cancer is acute myeloid leukaemia (AML).

In yet another aspect, provided are kits for treating a cancer in a subject, the kit comprising: (a) a single dose or multiple doses of an innate immune modifier; (b) a single dose or multiple doses of an immune checkpoint inhibitor; (b) a single dose or multiple doses of a T-cell stimulator, and (d) instructions for using said innate immune modifier, said immune checkpoint inhibitor and said T-cell stimulator according to the methods described herein.

In some embodiments of the kit, (a), (b) and (c) are provided in a form or forms suitable for sequential, separate and/or simultaneous administration.

Additional embodiments of the methods, combinations, compositions, kits and the like will be apparent from the following description, examples, and claims. As can be appreciated from the foregoing and following description, each and every feature described herein, and each and every combination of two or more of such features, is included within the scope of the present disclosure provided that the features included in such a combination are not mutually inconsistent. In addition, any feature or combination of features may be specifically excluded from any embodiment.

### BRIEF DESCRIPTION OF THE FIGURES

FIG. 1 is a plot of mean tumor volume versus time after treatment in mice treated with various combinations of talabostat mesylate, a PD-1 antagonist (anti-PD-1 antibody), and (2,7-(bis-methoxyPEG_{10kD}-carboxyamide)(9H-fluorene-9-yl)methyl N-carbamate)_{6avg}interleukin-2 (also referred to herein as “RSLAIL-2”), and each of the single agents, as evaluated until day 28 in the Pan02 syngeneic mouse model of pancreatic carcinoma as described in Example 1. Group 1=vehicle control, Group 2=talabostat mesylate (20 mcg qd), Group 3=RSLAIL-2 (0.8 mg/kg; q9d), Group 4=PD-1 antagonist (10 mg/kg biw), Group 5=talabostat mesylate (20 mcg qd) and RSLAIL-2 (0.8 mg/kg; q9d), Group 6=talabostat mesylate (20 mcg qd) and PD-1 antagonist (10 mg/kg biw), Group 7=RSLAIL-2 (0.8 mg/kg; q9d) and PD-1 antagonist (10 mg/kg biw), and Group 8=talabostat mesylate (20 mcg qd), RSLAIL-2 (0.8 mg/kg; q9d), and PD-1 antagonist (10 mg/kg biw). These data illustrate the notable anti-tumor effects of various exemplary combinations, and in particular, the pronounced effect of all three components in combination, showing complete regression of the implanted tumor (Group 8). Tumor size was measured up to Day 29 after inoculation. The triple combination (Group 8) shows a p* value <0.001 as compared to talabostat mesylate and PD-1 antagonist (Group 6) as well as single agents (Groups 2 and 4, respectively). The triple combination (Group 8) shows a p # value <0.05, when compared to the talabostat combinations with PD-1 antagonist (Group 6) and RSLAIL-2 (Group 5) as well as the RSLAIL-2 combination with PD-1 antagonist (Group 7) and the single agent RSLAIL-2 (Group 3). The combination of talabostat mesylate and PD-1 antagonist (Group 6) shows a p⁺ value <0.05 as compared to PD-1 antagonist alone (Group 4)

FIG. 2A is a plot of mean tumor volume versus days following treatment in mice treated with various combinations of talabostat mesylate, a PD-1 antagonist, and (2,7-(bis-methoxyPEG_{10kD}-carboxyamide)(9H-fluorene-9-yl)methyl N-carbamate)_{6avg}interleukin-2 (“RSLAIL-2”), and each of the single agents, as evaluated in the Pan02 syngeneic mouse model of pancreatic carcinoma (Phase I, Example 1, also shown in FIG. 1). Dosing was stopped at Day 28 after tumor inoculation. All Group 8 mice treated with the combination remained tumor-free. Also shown in the plot are the results of a Phase 2 study in which the mice of Group 8 were re-challenged with a second inoculation of Pan02 tumor cells (3×10⁶), as was a control group of treatment naïve mice (Group 9) as described in Example 2. While the control group of mice showed notable tumor growth, 5 of 6 re-challenged mice of Group 8 remained tumor-free up until at least Day 285, indicating that the Phase 1 treatment had stimulated anti-tumor immunity. FIG. 2B is a plot of body weight (grams) versus days following treatment for the mice in the various treatment groups of the Phase I study (Examples 1). FIG. 2C is a plot of mean tumor volume versus days following treatment for treatment Group 8 and Group 9. Triangles show naïve mice treated with tumor. Asterisks show Group 8 rechallenged mice, including the sole mouse that grew a tumor.

FIG. 3A contains immunohistochemistry (IHC) images of FAP (Fibroblast Activation Protein) expression in tumor samples obtained from animals sacrificed 3 days (i.e. Day 8 after tumor inoculation) after receiving immunotherapeutic treatment as described in Example 3 (Groups 1, 5, 6, and 8). The images show a reduction in FAP expression in tumors treated with the illustrative therapeutic triple combination (i.e., talabostat mesylate, RSLAIL-2 and a PD-1 antagonist).

FIG. 3B is a bar graph providing quantitative analyses of immunohistochemistry (IHC) images of the tumor samples described above (for Groups 1-8), and analysed by optical density (OD) for FAP expression as described in Example 3. The graph illustrates reduction in FAP+ cells due to treatment with the triple combination (talabostat mesylate, RSLAIL-2, and a PD-1 antagonist).

FIG. 4A provides immunohistochemistry (IHC) images of neutrophils (Ly6G+ cells) from tumor samples from the animals sacrificed 3 days after receiving treatment (Groups 1, 2, 4 and 8), as described in Example 3. The images illustrate an increase in Ly6G+ cells resulting from treatment with the triple combination (talabostat mesylate, RSLAIL-2, and a PD-1 antagonist).

FIG. 4B is a bar chart that provides quantitative analyses of immunohistochemistry (IHC) images of the tumor samples described above (Groups 1-8) analyzed for percentage of neutrophils (Ly6G+ cells) as described in Example 3. The graph illustrates an increase in Ly6G+ cells in tumors treated with the triple combination (talabostat mesylate, RSLAIL-2, and PD-1 antagonist).

FIG. 4C is an enlarged view of the IHC image (in FIG. 4A, Group 8) confirming neutrophil (Ly6G+ cells) influx in tumors of mice 3 days after receiving treatment with the triple combination (talabostat mesylate, RSLAIL-2, and a PD-1 antagonist).

FIG. 4D provides immunohistochemistry (IHC) images of CD8+ lymphocyte infiltration in tumor samples obtained from animals that were sacrificed 3 days after receiving treatment (Groups 1, 5, 6, and 8) as described in Example 3. The images indicate an increase in CD8+ lymphocyte infiltrates in tumors from mice treated with the triple combination (talabostat mesylate, RSLAIL-2 and a PD-1 antagonist).

FIG. 5 provides immunohistochemistry (IHC) images from tumor samples from animals that were sacrificed 3 days after receiving treatment (Groups 1, 4, 6, and 8), as described in Example 3. The images illustrate a decrease in numbers of tumor cells (H & E staining) in the tumor samples obtained from animals treated with the exemplary triple combination (talabostat mesylate, RSLAIL-2, and a PD-1 antagonist).

FIG. 6A provides the results of a multiplex assay for cytokines/chemokines (MILLIPLEX® MAP, Merck Millipore) on plasma collected before (pre-treatment) and 7 days following administration of the triple combination to mice (post-treatment) with Pan02 tumors as described in Example 4. Administration of the triple combination (talabostat mesylate, RSLAIL-2 and a PD-1 antagonist) resulted in an increase in pro-inflammatory cytokines (IL-6, IL-12p40, Tumor Necrosis Factor (TNF) alpha, and RANTES).

FIG. 6B provides the results of a multiplex assay for cytokines/chemokines (MILLIPLEX® MAP, Merck Millipore) on plasma collected before (pre-treatment) and 7 days following administration of the triple combination to mice (post-treatment) with Pan02 tumors as described in Example 4. Administration of the triple combination (talabostat mesylate, RSLAIL-2 and a PD-1 antagonist) resulted in a significant increase in GM-CSF (immune-stimulatory cytokine) in plasma.

FIG. 6C provides the results of a multiplex assay for cytokines/chemokines (MILLIPLEX® MAP, Merck Millipore) on plasma collected before (pre-treatment) and 7 days following administration of the triple combination to mice (post-treatment) with Pan02 tumors as described in Example 4. The data shows that the triple combination (talabostat mesylate, RSLAIL-2 and a PD-1 antagonist) resulted in a decrease in CXCL5 (Chemokine (C-X-C motif) ligand), a protein that is involved in proliferation, migration and invasion.

FIG. 6D provides the results of a multiplex assay for cytokines/chemokines (MILLIPLEX® MAP, Merck Millipore) on plasma collected before (pre-treatment) and 7 days following administration of the triple combination to mice (post-treatment) with Pan02 tumors as described in Example 4. This data shows that administration of the triple combination (talabostat mesylate, RSLAIL-2 and a PD-1 antagonist) resulted in an increase in cytokines inducing T-cell migration (monokine induced by gamma interferon (MIG), and macrophage inflammatory proteins (MIP1-beta)).

FIG. 6E provides the results of a multiplex assay for cytokines/chemokines (MILLIPLEX® MAP, Merck Millipore) on plasma collected before (pre-treatment) and 7 days following administration of the triple combination to mice (post-treatment) with Pan02 tumors as described in Example 4. This data shows that administration of the triple combination (talabostat mesylate, RSLAIL-2 and a PD-1 antagonist) resulted in an increase in cytokines inducing memory T cells (IL-7 and IL-15).

FIG. 7 is a bar graph showing the results of FACS analyzed data for splenocytes from mice (treated with triple combination described in Example 2) and sacrificed on Day 289 following a second re-challenge with Pan02 tumor cells. The CD62L-/CD44hi response for Group A confirms the development of a CD8+ effector memory T cell response as described in Example 5. In contrast, the naïve sets of mice inoculated with the Pan02 tumor cells and with no inoculum showed no significant generation memory markers.

FIG. 8A is a plot of mean tumor volume versus days following tumor inoculation for mice treated with the triple combination (talabostat mesylate, RSLAIL-2 and a PD-1 antagonist) in a WEHI 164 mouse sarcoma model as described in Example 6, and illustrates complete tumor disappearance in treated mice. (* shows re-challenge at Day 137).

FIG. 8B is a plot of mean tumor volume versus days following tumor re-challenge for mice treated with the triple combination (talabostat mesylate, RSLAIL-2 and a PD-1 antagonist) in a WEHI 164 mouse sarcoma model as described in Example 6. To assess the formation of a memory anti-tumor response, the group was re-challenged with WEHI 164 tumor cells (1×10⁶). FIG. 8A* shows re-challenge at Day 137. The treated mice showed resistance to tumor growth, while the group of treatment naïve mice inoculated with WEHI 164 tumor cells experienced tumor growth.

FIG. 9A is a plot of mean tumor volume versus days following tumor inoculation for mice treated with the triple combination (talabostat mesylate, RSLAIL-2 and a PD-1 antagonist) in a MC38 mouse colon cancer model. The plot shows complete tumor regression (elimination) following treatment with the triple combination as described in detail in Example 7.

FIG. 9B is a plot of mean tumor volume versus days following tumor re-challenge for mice treated with the triple combination (talabostat mesylate, RSLAIL-2 and a PD-1 antagonist) in an MC38 mouse colon cancer model as described in Example 7. All treated rechallenged mice demonstrated resistance to tumor growth, in contrast to the naïve set of mice inoculated with the MC 38 tumor cells, and in which tumor growth was observed. These results indicate that a memory immune response was induced in the triple-combination treated mice. FIG. 9A* shows re-challenge at Day 136.

### DETAILED DESCRIPTION

### Abbreviations

AML: Acute myeloid leukemia

B.ID: Bis in die (i.e. twice daily)

BTLA: B- and T-lymphocyte attenuator

BIW: Twice a week

CTLA4: Cytotoxic T-lymphocyte associated protein 4

CD: Cluster of differentiation

CXCL: Chemokine (C-X-C motif) ligand

CLL: Chronic lymphocytic leukemia

DPP: Dipeptidyl peptidase

DMEM: Dulbecco's Modified Eagle Medium

FAP: Fibroblast activation protein

GM-CSF: Granulocyte-macrophage colony-stimulating factor G-CSF:

HBSS: Hank's Balanced Salt Solution

IL: Interleukin

IHC: Immunohistochemistry

PD-1: Programmed Cell Death 1

PDL-1: Programmed death-ligand 1

PDL-2: Programmed death-ligand 2

MIG: Monokine induced by gamma interferon

MIP: Macrophage Inflammatory Proteins

NK: Natural killer

OD: Optical density

Q.D: Quaque die (i.e. once a day)

Q3W: Every three weeks

Q2W: Every two weeks

Q9D: Every 9^{th} day

TNF: Tumor necrosis factor

### Definitions

In describing and claiming certain features of this disclosure, the following terminology will be used in accordance with the definitions provided below unless indicated otherwise.

As used in this specification, the singular forms “a,” “an,” and “the” include plural referents unless the context clearly dictates otherwise.

As used herein, the term “subject” refers to a living organism suffering from or prone to a condition that can be prevented or treated by administration of a composition or combination as provided herein, such as a cancer, and includes both humans and animals. Subjects include, but are not limited to, mammals (e.g., murines, simians, equines, bovines, porcines, canines, felines, and the like). Typically, the subject is a human.

As used herein, the term “cancer” can be used interchangeably with “tumor” (that is to say, reference to a tumor as used herein is in reference to a cancerous tumor). The term “cancer” refers to a wide variety of types of cancer, including both solid tumors and non-solid tumors such as leukemia and lymphoma. Cancers include carcinomas, sarcomas, myelomas, lymphomas, and leukemia, and each can be treated in accordance with the combinations and methods provided herein, including those cancers which have a mixed type.

As used herein, the term “treatment”, “treating” and the like include both treatment to effect an anti-cancer response and to maintain anti-cancer immunity following cancer regression.

As used herein, the phrase “effective amount” refers to the quantity of a component or of a combination, which is sufficient to yield a desired therapeutic response, for example, a reduction in tumor growth or in tumor size, without undue adverse side effects (such as, for example, toxicity, irritation, or allergic response) commensurate with a reasonable benefit/risk ratio when used in the manner of this disclosure. A particular therapeutically effective amount will vary with factors such as the particular condition being treated, the physical condition of the patient, the type of mammal or animal being treated, the duration of the treatment, the nature of concurrent therapy (if any), and the specific formulations employed and the structures and types of compounds being administered.

As used herein, the term “innate immunity modifier” refers to a small molecule or an antibody that, when specifically bound with a cognate binding partner present on innate immune cells, e.g., macrophages, dendritic cells, neutrophils, natural killer cells and like, leads to activation of the innate immune system (e.g., pro-inflammatory cytokines), preferably to provide an anticancer effect.

As used herein, the term “immune checkpoint inhibitor” or “checkpoint inhibitor” refers to a compound that inhibits the immune checkpoint involved in immune escape as harnessed by the cancer progressing tumor microenvironment.

As used herein, a “T-cell stimulator” refers to an antibody, a small molecule, a cytokine (optionally in polymer-modified form) and/or a ligand that, when specifically bound with a cognate binding partner on a T-cell, mediates a response by the T-cell, including, but not limited to, activation, initiation of an immune response, inhibition of tumor proliferation, cytokine production and the like.

“PEG” or “polyethylene glycol,” as used herein, is meant to encompass any water-soluble poly(ethylene oxide). Unless otherwise indicated, a “PEG polymer” or a polyethylene glycol is one in which substantially all (preferably all) monomeric subunits are ethylene oxide subunits, though, the polymer may contain distinct end capping moieties or functional groups, e.g., for conjugation. PEG polymers for use in the present invention will comprise one of the two following structures: “—(CH₂CH₂O)ₙ—” or “—(CH₂CH₂)ₙ₋₁CH₂C₂—,” depending upon whether or not the terminal oxygen(s) has been displaced, e.g., during a synthetic transformation. For the PEG polymers, the variable (n) ranges from about 3 to 4000, and the terminal groups and architecture of the overall PEG can vary.

A “PEGylated IL-2” or “PEG-IL-2” is an IL-2 molecule (e.g. recombinant human IL-2) having one or more polyethylene glycol molecules covalently attached to one or more than one amino acid residue of the IL-2 protein, typically via a linker.

As used herein, the term “pharmaceutically acceptable excipient” refers to a non-toxic, inert solid, semi-solid or liquid filler, diluent, encapsulating material or formulation auxiliary of any type. A pharmaceutically acceptable excipient is any excipient, which is relatively non-toxic and innocuous to a patient at concentrations consistent with effective activity of the active ingredient so that any side effects ascribable to the excipient do not vitiate the beneficial effects of the active ingredient. Pharmaceutically acceptable excipients are for example carriers, diluents, disintegrants, binders, lubricants, fillers, plasticizers, surfactants and wetting agents, film-forming agents and coating materials, and colouring agents, for example pigments.

As used herein, the expressions “concurrent administration”, “simultaneous administration” or “administered simultaneously”, mean that the compounds are administered at the same point in time or immediately following one another. In the latter case, the compounds are administered at times sufficiently close that the results observed are essentially indistinguishable from those achieved when the compounds are administered at the same point in time.

“Dipeptidyl peptidase (DPP)” refers to a class of enzymes encoded by DPP gene (classified under EC 3.4.14). There are 9 types of DPP genes are known to date. These include Cathepsin C (DPP-1), DPP-2, DPP-3, DPP-4, DPP-6, DPP-7, DPP-8, DPP-9 and DPP-10. The DPP also includes fibroblast activation protein (FAP).

The terms “selective dipeptidyl peptidases” and “DPP-8/DPP-9/FAP” refer to a subset of DPP enzymes or genes containing one or more of DPP-8, DPP-9 and FAP. The term “selective dipeptidyl peptidase inhibitor” also referred to interchangeably herein as a “DPP8/DPP9/FAP inhibitor”, is a compound that selectively inhibits DPP8 and/or DPP9, FAP or DPP8, DPP9 and FAP in preference to other members of the DPP class of enzymes.

As used herein, the terms “Programmed Death 1,” “Programmed Cell Death 1,” “Protein PD-1,” “PD-1,” PD1,” “PDCD1,” “hPD-1” and “hPD-I” are used interchangeably, and include variants, isoforms, species homologs of human PD-1, and analogues having at least one common epitope with human PD-1.

As used herein, the terms “Programmed Cell Death 1 Ligand 1”, “PDL-1”, “PDL1”, “PDCD1L1”, “PDCD1LG1”, “CD274”, “B7 homolog 1”, “B7-H1”, “B7-H”, and “B7H1” are used interchangeably, and include variants, isoforms, species homologs of human PDL-1, and analogues having at least one common epitope with human PDL-1. The term “pharmaceutically acceptable salt” refers to salts derived from a variety of organic and inorganic counter ions well known in the art. Reference to compounds herein is meant to encompass pharmaceutically acceptable salt forms, as appropriate. Pharmaceutically acceptable acid addition salts may be formed with inorganic acids and organic acids. For reviews of suitable salts, see, e.g., Berge, et al., J. Pharm. Sci. 66:1-19 (1977) and Remington: The Science and Practice of Pharmacy, 20th Ed., ed. A. Gennaro, Lippincott Williams & Wilkins, 2000. Non-limiting examples of suitable acid salts includes: hydrochloric acid, hydrobromic acid, sulfuric acid, nitric acid, phosphoric acid, acetic acid, propionic acid, glycolic acid, pyruvic acid, oxalic acid, maleic acid, malonic acid, succinic acid, lactate acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Non-limiting examples of suitable base salts includes: sodium, potassium, lithium, ammonium, calcium, magnesium, iron, zinc, copper, manganese, aluminum, primary, secondary, and tertiary amines, substituted amines including naturally occurring substituted amines, cyclic amines, basic ion exchange resins, and the like, specifically such as isopropylamine, trimethylamine, diethylamine, triethylamine, tripropylamine, and ethanolamine.

Compounds described herein, when containing one or more chiral centers, are meant to encompass all stereoisomeric forms and mixtures thereof, including enantiomers, diastereoisomers, racemic mixtures, mixtures of enantiomers where one enantiomer is present in enantiomeric excess, and the like.

Herein, reference to administration of a “combination” refers to the simultaneous, separate or sequential administration of the components of the combination. For example, administration of components of a combination may refer to simultaneous administration (separately or together as part of a single pharmaceutical formulation). In yet another instance, administration of various components of a “combination” may refer to separate administration of each of the components, when administered separately, each of components are prepared as separate pharmaceutical compositions suitable for administration via appropriate administration routes. In yet a further example, administration of a “combination” may refer to sequential administration of each of the components of the combination and in any order. Where the administration is sequential or separate, a delay in administering a second or third or, for example, fourth component should be such that the agents are present in the body so as to produce a beneficial or synergistic effect of the combination.

“Substantially” or “essentially” means nearly totally or completely, for instance, 95% or greater, more preferably 97% or greater, still more preferably 98% or greater, even more preferably 99% or greater, yet still more preferably 99.9% or greater, with 99.99% or greater being most preferred of some given quantity.

“Optional” or “optionally” means that the subsequently described circumstance may but need not necessarily occur, so that the description includes instances where the circumstance occurs and instances where it does not.

A “small molecule” as used herein refers to an organic compound typically having a molecular weight of less than about 1000.

### Combination Components

Overview

In an effort to address as least some of the shortcomings associated with current anti-cancer immunotherapies, the present disclosure provides improved immunotherapeutic modalities, combinations and methods that utilize both the adaptive arm of the immune system and an innate arm of the immune system for treating cancer. The illustrative combinations described herein comprising an innate immune modifier, an immune checkpoint inhibitor, and a T-cell stimulator, facilitate notably enhanced immune system-based attacks on cancerous tumors, and are shown in representative animal models to provide surprisingly robust anti-tumor effects (such as, for example, complete tumor regression) as well as long-term anti-tumor immunity, among other things. See, for example, supporting Examples 1-7 herein. The present combination of agents is effective to result in significant immune activation that appears to arise as a result of each of the single agent components functioning in a non-redundant and complementary fashion.

These and related features of the subject immunotherapeutic combination will now be more fully described.

Innate Immunity Modifier

As described above, the present combinations comprise, as one component, an innate immunity modifier. One preferred class of innate immunity modifiers inhibits one or more of DPP 8/9 and FAP and is referred to herein as a “selective dipeptidyl peptidase inhibitor”.

The innate immune modifier may, for example, be a small molecule, antibody, nanobody, engineered peptide, engineered protein, vaccine, or siRNA, and is preferably a small molecule.

One preferred small molecule selective dipeptidyl peptidase inhibitor is talabostat (PubChem ID: 6918572), or a pharmaceutically acceptable salt thereof, such as, for example, talabostat mesylate (PubChem CID: 1152248). Talabostat, also known as PT-100 (Val-boro-pro; L-valinyl-L-boroproline), is disclosed in PCT Appl. Publication No. WO1989003223 (CAS registry number 149682-77-9). The IUPAC name of talabostat is [(2R)-1-[(2S)-2-amino-3-methylbutanoyl]pyrrolidin-2-yl]boronic acid. Talabostat has two chiral centers, and may be used as the free base or as a pharmaceutically acceptable salt, in any of its enantiomeric or diastereomeric forms, including mixtures thereof. Talabostat or a pharmaceutically acceptable salt thereof can also exist in both its non-cyclized and cyclic forms (RJ Snow et al., J. Am. Chem. Soc., 1994, 116 (24), pp 10860-10869). Other pharmaceutically acceptable salts include, for example, those prepared from typical inorganic acids such as hydrochloric, hydrobromic, hydroiodic, nitric, sulfuric, phosphoric, hypophosphoric, and the like, as well as those prepared from organic acids, such as for example, aliphatic mono and dicarboxylic acids, phenyl substituted alkanoic acids, hydroxyalkanoic and hydroxyl alkandioic acids, aromatic acids, aliphatic (mesylate) and aromatic sulfonic acids, and any suitable form of talabostat may be used in the combinations provided herein and the disclosure is not limited in this regard. A preferred salt form of talabostat is the mesylate salt. Talabostat mesylate has a CAS registry number of 150080-09-4 and an IUPAC name as follows: [(2R)-1-[(2S)-2-amino-3-methylbutanoyl]pyrrolidin-2-yl]boronic acid; methanesulfonic acid.

Various other small molecules are also encompassed in the scope of the present disclosure, such as, for example, analogs and prodrugs of talabostat, as well as talabostat-like compounds. Illustrative compounds encompass those described in at least the following documents. EP Patent No. 2,782,994 discloses talabostat analogs, such as, for example, ARI-4175 and related compounds. PCT Appl. Publication No. WO2003092605 discloses prodrugs of talabostat, such as, for example, cyclohexyl(glycinyl)-prolinyl-valinyl-L-boroproline. PCT Appl. Publication Nos. WO2018049014 and WO2018049008 disclose various compounds of the boro-pro class, and other dipeptides, and are herein referred to as talabostat-like boro-pro compounds.

In some embodiments, the innate immune modifier is an antibody, such as an antibody that inhibits FAP. The FAP inhibitor may, in some instances, be a FAP monoclonal antibody, such as for example, sibrotuzumab. Other FAP inhibitors include, but are not limited to ARI-3099 (N-(pyridine-4-carbonyl)-d-Ala-boroPro) as disclosed in Sarah E. Poplawski et al., 2013, Vol. 56(9) pp. 3467-3477; ARI-3996 as disclosed in U.S. Patent Appl. Publication No. 20140255300; MIP-1231 (MIP-1232 or MIP-1233) as disclosed in U.S. Patent Appl. Publication No. 20100098633; (4-quinolinoyl)-glycyl-2-cyanopyyrolidines as disclosed by Koen Jansen et al., 2013, Vol. 4 (5), Page no. 491-496; (2S)-1-(2-(1-Napthoylamino)acetyl)pyrroline-2-carbonitrile as disclosed in U.S. Pat. No. 8,183,280; (S)-A-(2-(2-cyano-4,4-difluoropyrrolidin-1-yl)-2-oxoethyl)-1-naphthamide and other related derivatives as disclosed in PCT Appl. Publication No. WO2013107820; (2S)-1-((2 S)-2-(2-Methoxybenzoylamino)-3-methylpentanoyl) pyrrolidine-2-carbonitrile and other related derivatives as disclosed in U.S. Patent Appl. Publication No. 20120053222; Ac-Gly-BoroPro as disclosed by Conrad Yap Edosada et al. 2006, Vo. 281(11) page no. 7437-7444; Substituted 4-carboxylmethyl pyroglutamic acid diamides as disclosed in Ting-yueh Tsai et al., 2010, Vol. 53(18), 6572-6583; GEH200200 as disclosed by P. Iveson et al., 2014, Vol. 41(7), 620; UAMC-1110 as disclosed in U.S. Pat. No. 9,346,814; as well as FAP inhibitors also disclosed in PCT Appl. Publication No. WO2002038590, U.S. Pat. No. 7,399,869; and 7,998,997.

Additional FAP inhibitors include FAP-α antibodies such as described in U.S. Pat. No. 8,568,727, European Patent No. 1,268,550, U.S. Pat. Nos. 8,999,342 and 9,011,847. Additional illustrative inhibitors include bispecific antibodies of FAP with DR-5 such as disclosed in U.S. Patent Appl. Publication Nos. 20140370019 and 20120184718. Also suitable for use are chimeric antigen receptor and FAP combinations such as disclosed in U.S. Patent Appl. Publication No. 20140099340.

In other aspects, the anti-FAP antibody may be a nanobody. Nanobody technology was developed from the discovery that antibodies from camels and llamas (Camelidae, camelids) have heavy chains but no light chains. The antigen-binding site of such antibodies is one single domain and may be referred to as VHH. See, e.g., U.S. Pat. Nos. 5,800,988 and 6,005,079 and PCT Appl. Publication Nos. WO 94/04678, WO 94/25591 and European Publ. No. EP 2673297 which are incorporated by reference.

Immune Checkpoint Inhibitors

In one or more particular aspects, the immune checkpoint inhibitor is an antibody or a small molecule. For example, the antibody may be directed against PD-1, PDL-1, or CTLA4. For example, the antibody may be selected from one or more of TECENTRIQ® (atezolizumab), KEYTRUDA® (pembrolizumab), BAVENCIO® (avelumab), YERVOY® (ipilimumab) and OPDIVO® (nivolumab). In some embodiments, the immune checkpoint inhibitor is a PD-1 axis antagonist or a CTLA4 antagonist.

### PD-1 Axis Antagonist:

Suitable for use in the combinations provided herein are PD-1 axis antagonists including PD-1 antagonists (for example an anti-PD-1 antibody), PDL-1 antagonists (for example an anti-PDL-1 antibody) and PDL-2 antagonists (for example an anti-PDL-2 antibody).

The complete human PD-1 sequence can be found under GenBank Accession No. U64863. In particular aspects, the PD-1 antagonist binds the PD-1 protein of SEQ ID NO: 1 (uniprot ID Q15116).

The protein programmed death 1 (PD-1) is an inhibitory member of the CD28 family of receptors that also includes CD28, CTLA-4, ICOS and BTLA. Two ligands for PD-1 have been identified, PDL-1 and PDL-2, that have been shown to downregulate T-cell activation upon binding to PD-1 (Freeman et al. (2000) J Exp. Med. 192: 1027-34; Latchman et al. (2001) Nat Immunol. 2:261-8; Carter et al. (2002) Eur. J Immunol 32:634-43). Both PDL-1 and PDL-2 are B7 homologs that bind to PD-1, but do not bind to other CD28 family members.

A PD-1 axis antagonist for use in the combination therapies described herein bind to ligands of PD-1 and interfere with, reduce, or inhibit the binding of one or more ligands to the PD-1 receptor, or binds directly to the PD-1 receptor without engaging in signal transduction through the PD-1 receptor. The PD-1 axis antagonist binds to one or more ligands of PD-1 (e.g., PDL-1 and PDL-2) and reduces or inhibits the ligand(s) from triggering inhibitory signal transduction through PD-1. In one or more embodiments, the PD-1 axis antagonist binds directly to PDL-1, inhibiting or preventing PDL-1 from binding to PD-1, thereby blocking PD-1 inhibitory signal transduction.

In some embodiments, the antibody interfering with PD-1 is an anti-PD-1 antibody (e.g., a human antibody, a humanized antibody, or a chimeric antibody) such as described below. For example, suitable for use in the combinations disclosed herein is nivolumab (also known as Opdivo®, MDX-1106, MDX-1106-04, ONO-4538 or BMS-936558). Nivolumab is a fully humanized IgG4 (S228P) PD-1 antibody that selectively prevents interaction with PD-1 ligands (PD-L1 and PD-L2), thereby blocking the down-regulation of antitumor T-cell functions (U.S. Pat. No. 8,008,449; PCT Appl. Publication No. WO2006/121168; Wang et al, Cancer Immunol Res. 2:846-56 (2014); Topalian, S. L. et al, N Engl J Med 366.2443-2454 (2012); Topalian, S. L. et al, Current Opinion in Immunology 24:207-212 (2012); Topalian, S. L. et al, J Clin Oncol 31 (suppl):3002 (2013)). Nivolumab has been approved by the U.S. FDA for the treatment of patients with unresectable or metastatic melanoma, metastatic squamous non-small cell lung cancer, advanced renal cell carcinoma, and classical Hodgkin lymphoma.

In some other embodiments, the PD-1 antagonist is pembrolizumab (trade name KEYTRUDA®; also known previously as lambrolizumab, SCH-900475 and MK-3475) is a humanized monoclonal IgG4 kappa antibody directed against PD-1. Hamid, O. et al, N Engl J Med 369: 134-144 (2013). Pembrolizumab is described, for example, in U.S. Pat. Nos. 8,354,509 and 8,900,587 and PCT Application Publication No. WO2009/114335. Pembrolizumab has been approved by the U.S. FDA for the treatment of patients with advanced melanoma, non-small cell lung cancer, and head and neck squamous cell cancer. See, e.g., Poole, R. M., Drugs 74: 1973-1981 (2014). In a preferred embodiment, the anti-PD-1 antibody used in the methods (and kits) described herein is pembrolizumab or nivolumab Other PD-1 antagonists that can be employed in the therapeutic combinations described herein are disclosed in U.S. Pat. No. 8,609,089, US Patent Appl. Publication No. 20100028330, and/or in US Patent Appl. Publication No. 20120114649.

Additional PD-1 axis antagonists that may be used include, for example, atezolimumab (MDPL3280A or YW243.55.S70), a PDL-1 antagonist described in U.S. Pat. No. 8,217,149. MDX-1105 (also known as BMS-936559) a PDL-1 antagonist described in PCT Appl. Publication No. WO2007/005874, durvalumab (MEDI4736), a PDL-1 antagonist described in PCT Appl. Publication No. WO2011/066389 and US2013/0034559, avelumab (MSB0010718C), a PDL-1 antagonist described in U.S. Patent Appl. Publication No. 20140341917, and CA-170, a PDL-1 antagonist described in PCT Appl. Publication Nos. WO2015033301 and WO2015033299. In some embodiments, rather than using an antibody that targets PD-L1, a small molecule that targets PD-L1 can also be used in the methods and kits of the invention. For example, CA-170 in development by Curis, Inc., is an orally available small molecule that selectively targets and inhibits PD-Ll, PD-L2, and V-domain immunoglobulin suppressor of T-cell activation (VISTA) checkpoint regulators of immune activation. Curis is currently investigating CA-170 in a Phase 1 trial in patients with advanced solid tumors and lymphomas. See www.clinicaltrials.gov (NCT02812875).

An additional checkpoint inhibitor that may be used is AMP-224 (also known as B7-DCIg), a PDL-2-Fc fusion soluble receptor described in PCT Appl. Publication Nos. WO2010/027827 and WO2011/066342.

Other PD-1 antagonists include BCD100, 1B1308, camrelizumab, JNJ63723283, JS001, spartalizumab, cemiplimab and tislelizumab and combination thereof.

In one or more embodiments, the PD-1 antagonist is selected from the group consisting of ANA011, AUNP-12, BGB-A317, KD033, pembrolizumab, MCLA-134, mDX400, MEDI00680, muDX400, nivolumab, PDR001, PF-06801591, REGN-2810, SHR-1210, STI-A1110, TSR-042, ANB011, 244C8, 388D4, TSR042, BCD100, camrelizumab, JNJ63723283, JS001, spartalizumab, cemiplimab, tislelizumab, and XCE853 and combination thereof. PD-1 antagonists (e.g. anti-PD-1 antibodies) may, for example, be procured from BPS Biosciences, Bioxcell or other commercial sources.

In one or more embodiments, the PDL-1 antagonist is selected from the group consisting ofavelumab, BMS-936559, CA-170, durvalumab, MCLA-145, SP142, STI-A1011, STIA1012, STI-A1010, STI-A1014, A110, KY1003 and atezolizumab and combinations thereof. Preferably the PDL-1 antagonist is avelumab, durvalumab or atezolizumab.

In some additional embodiments, the PDL-2 antagonist is selected from the group consisting of AMP-224 and rHIgM12B7 and a combination thereof.

The antibody or an antigen binding fragment thereof may be made using methods known in the art, for example, by a process comprising culturing a host T-cell containing nucleic acid encoding any of the previously described PD-1, PDL-1, or PDL-2 antibody or antigen-binding fragment in a form suitable for expression, under conditions suitable to produce such antibody or fragment, and recovering the antibody or fragment.

### CTLA4 Antagonists

Suitable CTLA4 antagonist agents for use in the combination products described herein, include, without limitation, anti-CTLA4 antibodies, human anti-CTLA4 antibodies, mouse anti-CTLA4 antibodies, mammalian anti-CTLA4 antibodies, humanized anti-CTLA4 antibodies, monoclonal anti-CTLA4 antibodies, polyclonal anti-CTLA4 antibodies, chimeric anti-CTLA4 antibodies, MDX-010 (ipilimumab), tremelimumab, anti-CD28 antibodies, anti-CTLA4 adnectins, anti-CTLA4 domain antibodies, single chain anti-CTLA4 fragments, heavy chain anti-CTLA4 fragments, light chain anti-CTLA4 fragments, inhibitors of CTLA4 that agonize the co-stimulatory pathway, the antibodies disclosed in PCT Appl. Publication No. WO 2001/014424, the antibodies disclosed in PCT Appl. Publication No. WO 2004/035607, the antibodies disclosed in U.S. Patent Appl. Publication No. 2005/0201994, and, for example, the antibodies disclosed in European Patent No. 1212422 B. Additional exemplary anti-CTLA-4 antibodies are described in U.S. Pat. Nos. 5,811,097, 5,855,887, 6,051,227, and 6,984,720; in PCT Appl. Publication Nos. WO 01/14424 and WO 00/37504; and in U.S. Patent Appl. Publication Nos. US2002/0039581 and US2002/086014. Other anti-CTLA-4 antibodies that can be used in a method or combination as described herein include, for example, those disclosed in: PCT Appl. Publication No. WO 98/42752; U.S. Pat. Nos. 6,682,736 and 6,207,156; Hurwitz et al., Proc. Natl. Acad. Sci. USA, 95(17): 10067-10071 (1998); Camacho et al., J. Clin: Oncology, 22(145): Abstract No. 2505 (2004) (antibody CP-675206); Mokyr et al., Cancer Res., 58:5301-5304 (1998), and U.S. Pat. Nos. 5,977,318, 6,682,736, 7,109,003, and 7,132,281.

A preferred clinical anti-CTLA-4 antibody is human monoclonal antibody 10DI (also referred to as MDX-010 or ipilimumab, available from Medarex, Inc., Bloomsbury, N.J.), disclosed in PCT Appl. Publication No. WO 01/14424. In other embodiments, the anti-CTLA-4 antibody is tremelimumab. Other CTLA4 antagonist (anti-CTLA-4 antibody) may be selected from the group consisting of KAHR-102, AGEN1884, ABR002, and KN044 and combinations thereof.

### T-cell Stimulator

The combinations, compositions, methods and the like provided herein comprise a T-cell stimulator. In certain embodiments of the combinations and methods provided herein, the T-cell stimulator stimulates activity via the IL-2 receptor. Thus, for example, the T-cell stimulator may be an IL-2 receptor agonist. In some embodiments, the IL-2 receptor agonist is an interleukin-2. In some other embodiments, the T-cell stimulator is a CD122-biased agonist (IL-2Rβ biased agonist). For example, the IL-2 receptor agonist may be an interleukin-2 that is chemically modified, such as by PEGylation, and more particularly, by releasable PEGylation. An interleukin-2 receptor beta (IL-2Rβ) selective agonist (i.e., a CD122-biased agonist) is an agonist that has a greater affinity for binding to IL-2Rβ than to IL-2Rαβ. By way of example, it is possible to measure binding affinities relative to IL-2 as a standard using surface plasmon resonance (using, e.g., a system such as BIACORE™ T100). Generally, a CD122-biased agonist will possess an in vitro binding affinity for IL-2Rβ that is at least 5 times greater (more preferably at least 10 times greater) than the binding affinity for IL-2Rαβ in the same in vitro model. In this regard, (2,7-(bis-methoxyPEG_{10kD}-carboxyamide)(9H-fluorene-9-yl)methyl N-carbamate)_{6avg}interleukin-2, a CD-122 biased cytokine agonist in which recombinant human interleukin-2 (de-1-alanine, 125-serine), is N-substituted with an average of six [(2,7-bis{[methylpoly(oxyethylene)_{10kD}]carbamoyl}-9H-fluoren-9-yl)methoxy]carbonyl moieties at its amino residues (CAS No. 1939126-74-5) exhibits about a 60-fold decrease in affinity to IL-2Rax relative to IL-2, but only about a 5-fold decrease in affinity IL-2Rβ relative to IL-2.

In one or more embodiments, the T-cell stimulator is an IL-2Rβ selective agonist such as multi (2,7-(bis-methoxy PEG-carboxyamide)(9H-fluorene-9-yl)methyl N-carbamate)interleukin-2, and comprises compounds encompassed by the following Formula (I):

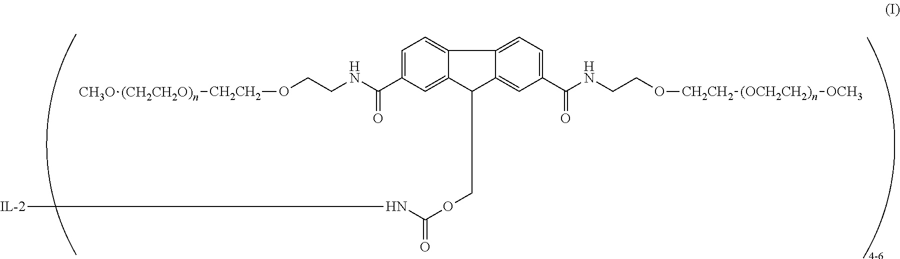

where IL-2 is an interleukin-2 (such as, for example, aldesleukin), and each “n” is independently an integer from about 3 to about 1000, or a pharmaceutically acceptable salt thereof. Representative ranges for each “n” include, for example, an integer from about 40 to about 550, or an integer from about 60 to about 500, or an integer from about 113 to about 400, or from 200-300. In certain embodiments, “n” in each of the polyethylene glycol chains is about 227 (i.e., where each polyethylene glycol chain extending from the central fluorenyl core has a weight average molecular weight of about 10,000 daltons, such that the weight average molecular weight of the overall branched PEG moiety is about 20,000 daltons), i.e., referred to herein as (2,7-(bis-methoxyPEG_{10kD}-carboxyamide)(9H-fluorene-9-yl)methyl N-carbamate)₄₋₆interleukin-2. In one or more embodiments, the value of “n” in each of the polyethylene glycol chains is substantially the same. In other embodiments, the two PEG chains extending from the central fluorenyl core have substantially the same weight average molecular weight.

In certain embodiments, multi(2,7-(bis-methoxyPEG-carboxyamide)(9H-fluorene-9-yl)methyl N-carbamate)interleukin-2 has a structure:

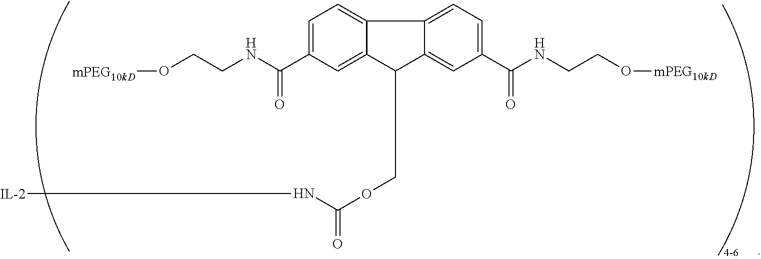

In other more particular embodiments, multi(2,7-(bis-methoxyPEG-carboxyamide)(9H-fluorene-9-yl)methyl N-carbamate)interleukin-2 has a structure:

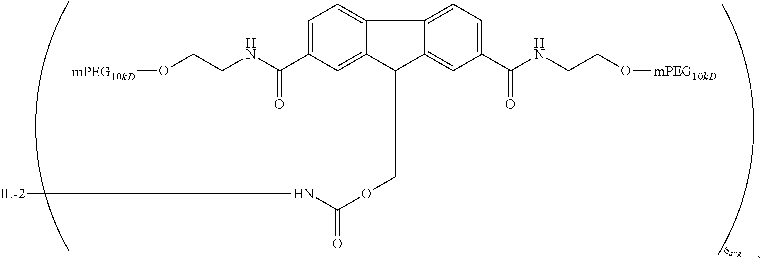

and is referred to herein as (2,7-(bis-methoxyPEG_{10kD}-carboxyamide)(9H-fluorene-9-yl)methyl N-carbamate)_{6avg}interleukin-2, or as RSLAIL-2.

The releasable PEG moiety comprised is based upon a 2,7,9-substituted fluorene with poly(ethylene glycol) chains extending from the 2- and 7-positions of the fluorene ring via amide linkages (fluorene-C(O)—NH—), to provide a branched PEG. The fluorenyl-based branched PEG moieties are releasably covalently attached to amino groups of the interleukin-2 moiety. The linkage between interleukin-2 amino groups and the fluorenyl-based branched PEG moiety is a carbamate linkage attached via a methylene group (—CH₂—) to the 9-position of the fluorene ring. Releasable PEGs having this general structure typically undergo a β-elimination reaction under physiological conditions to slowly release the PEG moieties that are covalently attached to the IL-2. It is believed that the PEG moieties release sequentially in vivo following administration.

In certain embodiments, the long acting IL-2RO-biased agonist of Formula (I) is comprised in a composition that contains no more than 10% (based on a molar amount), and in some embodiments no more than 5% (based on a molar amount), of compounds encompassed by the following formula:

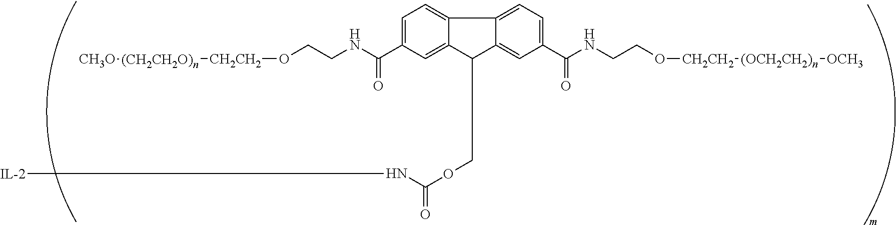

wherein IL-2 is an interleukin-2 such as aldesleukin, “m” (referring to the number of polyethylene glycol moieties attached to IL-2) is an integer selected from the group consisting of 1, 2, 3, 7 and >7, or pharmaceutically acceptable salts thereof.

In some embodiments, e.g., in reference to Formula (I), the long acting IL-2RD-biased agonist possesses on average about six branched polyethylene glycol moieties releasably attached to IL-2. In some further embodiments, e.g., in reference to Formula (I), the long acting IL-2RO-biased agonist is generally considered to be an inactive prodrug, i.e., that is inactive upon administration, and by virtue of slow release of the polyethylene glycol moieties in vivo following administration, provides active conjugated forms of interleukin-2 having fewer PEG moieties attached than in the conjugate that is initially administered. Multi(2,7-(bis-methoxyPEG-carboxyamide)(9H-fluorene-9-yl)methylN-carbamate)interleukin-2 preferentially activates the IL-2 receptor beta and gamma units over IL-2 receptor alpha, thereby providing a specific activation of the T effector cell and natural killer cell populations associated with the adaptive immune system over the immune suppressive T regulatory cells that also contain/express the IL-2 receptors, particularly the IL-2 R alpha.

Multi(2,7-(bis-methoxyPEG-carboxyamide)(9H-fluorene-9-yl)methyl N-carbamate)interleukin-2 can be prepared, e.g., as described in Example 1 in PCT Appl. Publication No. WO 2015/125159, by reaction of interleukin-2 (e.g., aldesleukin) with the PEG reagent, C2-PEG2-FMOC-NHS-20K (as described in PCT Appl. Publication No. WO 2006/138572).

Additional exemplary compositions of multi(2,7-(bis-methoxyPEG-carboxyamide)(9H-fluorene-9-yl)methylN-carbamate)interleukin-2 comprise compounds in accordance with Formula (I) wherein each fluorenyl-based PEG moiety has a weight average molecular weight in a range of from about 250 Daltons to about 90,000 Daltons. Additional suitable ranges include weight average molecular weights of each fluorenyl-based PEG moiety in a range selected from about 1,000 Daltons to about 60,000 Daltons, in a range of from about 5,000 Daltons to about 60,000 Daltons, in a range of about 10,000 Daltons to about 55,000 Daltons, in a range of from about 15,000 Daltons to about 50,000 Daltons, and in a range of from about 20,000 Daltons to about 50,000 Daltons.

Additional illustrative weight-average molecular weights for the fluorenyl-based polyethylene glycol moiety include about 200 Daltons, about 300 Daltons, about 400 Daltons, about 500 Daltons, about 600 Daltons, about 700 Daltons, about 750 Daltons, about 800 Daltons, about 900 Daltons, about 1,000 Daltons, about 1,500 Daltons, about 2,000 Daltons, about 2,200 Daltons, about 2,500 Daltons, about 3,000 Daltons, about 4,000 Daltons, about 4,400 Daltons, about 4,500 Daltons, about 5,000 Daltons, about 5,500 Daltons, about 6,000 Daltons, about 7,000 Daltons, about 7,500 Daltons, about 8,000 Daltons, about 9,000 Daltons, about 10,000 Daltons, about 11,000 Daltons, about 12,000 Daltons, about 13,000 Daltons, about 14,000 Daltons, about 15,000 Daltons, about 20,000 Daltons, about 22,500 Daltons, about 25,000 Daltons, about 30,000 Daltons, about 35,000 Daltons, about 40,000 Daltons, about 45,000 Daltons, about 50,000 Daltons, about 55,000 Daltons, about 60,000 Daltons, about 65,000 Daltons, about 70,000 Daltons, and about 75,000 Daltons. In some embodiments, the weight-average molecular weight of the polyethylene glycol polymer moiety is about 20,000 daltons.

Molecular weight in the context of a water-soluble polymer, such as PEG, can be expressed as either a number average molecular weight or a weight average molecular weight. Unless otherwise indicated, all references to molecular weight herein refer to the weight average molecular weight. Both molecular weight determinations, number average and weight average, can be measured using gel permeation chromatography or other liquid chromatography techniques. Other methods for measuring molecular weight values can also be used, such as the use of end-group analysis or the measurement of colligative properties (e.g., freezing-point depression, boiling-point elevation, or osmotic pressure) to determine number average molecular weight or the use of light scattering techniques, ultracentrifugation, or viscometry to determine weight average molecular weight. PEG polymers are typically polydisperse (i.e., number average molecular weight and weight average molecular weight of the polymers are not equal), possessing low poly-dispersity values of preferably less than about 1.2, more preferably less than about 1.15, still more preferably less than about 1.10, yet still more preferably less than about 1.05, and most preferably less than about 1.03.

The term “interleukin-2” or “IL-2” as used herein, e.g., in reference to multi(2,7-(bis-methoxyPEG-carboxyamide)(9H-fluorene-9-yl)methyl N-carbamate)interleukin-2, refers to a moiety having human IL-2 activity. Suitable proteins include proteins containing an amino acid sequence corresponding to any one of SEQ ID NOs: 1 through 4 described in U.S. Pat. No. 9,861,705. The term substantially homologous means that a particular subject sequence, for example, a mutant sequence, varies from a reference sequence by one or more substitutions, deletions, or additions, the net effect of which does not result in an adverse functional dissimilarity between the reference and subject sequences. For the purposes herein, sequences having greater than 95 percent homology (also referred to as sequence identity), equivalent biological activity (although not necessarily equivalent strength of biological activity), and equivalent expression characteristics are considered to be substantially homologous. For purposes of determining homology, truncation of the mature sequence should be disregarded. As used herein, the term “IL-2” includes such proteins modified deliberately, as for example, by site directed mutagenesis or accidentally through mutations. These terms also include analogs having from 1 to 6 additional glycosylation sites, analogs having at least one additional amino acid at the carboxy terminal end of the protein wherein the additional amino acid(s) includes at least one glycosylation site, and analogs having an amino acid sequence which includes at least one glycosylation site. The term includes both natural and recombinantly produced moieties. In addition, the IL-2 can be derived from human sources, animal sources, and plant sources. One exemplary IL-2 is recombinant IL-2 referred to as aldesleukin.

### IV. METHOD OF TREATMENT, DOSE AND ADMINISTRATION

As illustrated by the supporting animal model data provided herein, treatment of tumors in vivo with a combination comprising an exemplary innate immune modifier, an immune checkpoint inhibitor, and a T-cell stimulator, was effective to abolish the proliferative capacity of the tumor and produce a 100% anti-tumor effect. Therefore, the present disclosure provides a pharmaceutical combination with an enhanced immunogenic effect (in comparison to each of its components when administered singly, i.e., as a monotherapy) provided by its unique combination of components that overcomes the immune-resistance found in cancers, such as pancreatic cancer.

In one aspect, the present disclosure provides a method of treating cancer in a subject comprising administering to the subject an innate immunity modifier, an immune checkpoint inhibitor and a T cell stimulator. The innate immunity modifier, immune checkpoint inhibitor and T cell stimulator are each administered in an amount such that the combined therapy is effective to treat the cancer.

In one embodiment, the innate immunity modifier is a selective dipeptidyl peptidase inhibitor, preferably small molecule.

In another embodiment, the immune checkpoint inhibitor is a PD-1 axis antagonist.

In a further embodiment, the immune checkpoint inhibitor is a CTLA4 antagonist. In certain embodiments the T cell stimulator is an IL2Rβ selective agonist. In some embodiments, the T cell stimulator is a PEGylated IL-2. In some aspects, the T cell stimulator is a PEGylated IL-2 that is an IL2Rβ selective agonist. In a preferred embodiment, the present disclosure provides a method of treating cancer in a subject comprising administering to the subject a therapeutically effective amount of a selective dipeptidyl peptidase inhibitor, an immune checkpoint inhibitor and an IL2Rβ selective agonist, wherein
(i) the selective dipeptidyl peptidase inhibitor is talabostat or a pharmaceutically acceptable salt thereof,
(ii) the immune checkpoint inhibitor is a PD-1 axis antagonist or a CTLA4 antagonist, and
(iii) the IL2RP selective agonist comprises RSLAIL-2.

In another preferred embodiment, the selective dipeptidyl peptidase inhibitor is talabostat or a pharmaceutically acceptable salt thereof, e.g. talabostat mesylate.

In a further preferred embodiment, the immune checkpoint inhibitor is a PD-1 axis antagonist, e.g. a PD-1 antagonist (for example an anti-PD-1 antibody), or a PDL-1 antagonist, such as an antibody.

The present disclosure is also directed to a method of generating antitumor memory response in a subject in need thereof comprising administering combination comprising (a) an innate immune modifier, (b) an immune checkpoint inhibitor, and (c) a T-cell stimulator.

In one embodiment, (a), (b) and (c) above are administered to a subject at the same time (separately or together as part of a single pharmaceutical formulation), sequentially in any appropriate order or separately (e.g. intermittently), as a therapy to generate antitumor memory response. When administered separately, each of (a), (b) and (c) are prepared as separate pharmaceutical compositions suitable for administration via appropriate administration routes.

The present disclosure is also directed to a method of generating antitumor immune response in a subject in need thereof comprising administering combination comprising (a) an innate immune modifier, (b) an immune checkpoint inhibitor, and (c) a T-cell stimulator.

In one embodiment, (a), (b) and (c) above are administered to a subject at the same time (separately or together as part of a single pharmaceutical formulation), sequentially in any appropriate order or separately (e.g. intermittently), as a therapy to generate antitumor immune response. When administered separately, each of (a), (b) and (c) are prepared as separate pharmaceutical compositions suitable for administration via appropriate administration routes.

The present disclosure is also directed to a method of treating a patient suffering from a cancer, the method comprising the steps of administering to the patient: (a) an innate immunity modifier; (b) an immune checkpoint inhibitor and (c) a T-cell stimulator. Administration steps (a), (b) and (c) may be performed in any order (as well as simultaneously). In one or more embodiments, step (a) will be carried out before steps (b) and (c). In one or more embodiments, step (b) will be carried out before steps (a) and (c). In one or more embodiments, step (c) will be carried out before steps (a) (b). In one or more embodiments, steps (a), (b) and (c) will be carried out simultaneously. Further, in one or more embodiments, steps (a) and/or (b) and/or (c) will be administered repeatedly. In addition, one or more embodiments, steps (a) and (b) and (c) will be carried out only once.

The innate immune modifier, the immune checkpoint inhibitor and the T-cell stimulator can be administered accordingly to a suitable dosage and route (e.g., intravenous, intraperitoneal, intramuscular, intrathecal or subcutaneous). For example, the innate immune modifier, the immune checkpoint inhibitor and the T-cell stimulator can be simultaneously administered in a single formulation. Alternatively, the modifier, inhibitor and stimulator can be formulated for separate administration, wherein they are administered concurrently or sequentially.

In one embodiment, talabostat or a pharmaceutically acceptable salt thereof is co-administered with a PD-1 axis antagonist and a T-cell stimulator (e.g., an IL2Rβ biased agonist). In another embodiment, talabostat or a pharmaceutically acceptable salt thereof is administered independently from the administration of the PD-1 axis antagonist and T-cell stimulator (for example, an IL2Rβ selective agonist such as a PEGylated IL-2). In one embodiment, talabostat or a pharmaceutically acceptable salt thereof is administered first, followed by the T-cell stimulator (for example, an IL2Rβ selective agonist such as a PEGylated IL-2) and a PD-1 axis antagonist. In another embodiment, the T-cell stimulator (for example, an IL2Rβ selective agonist such as a PEGylated IL-2) and a PD-1 axis antagonist are administered first, followed by the administration of talabostat or a pharmaceutically acceptable salt thereof.

While particular methods disclosed herein involve administering all three therapeutic agents, the innate immune modifier (e.g. talabostat or a pharmaceutically acceptable salt thereof) and the T-cell stimulator (e.g., an IL2Rβ selective agonist such as a PEGylated IL-2) may be administered without including an immune checkpoint inhibitor as a part of the therapy. Optionally, a therapy may include initially administering all three agents at the start of a therapeutic regimen, and then switching, in a later cycle(s) of treatment, to administration of only an innate immune modifier and a T-cell stimulator. In other embodiments, an immune checkpoint inhibitor may be added to a therapeutic regimen already comprising an innate immune modifier and a T-cell stimulator.

Exemplary lengths of time associated with the course of therapy in accordance with the methods described herein include: about 3 days, about 4 days, about 5 days, about one week; about two weeks; about three weeks; about four weeks; about five weeks; about six weeks; about seven weeks; about eight weeks; about nine weeks; about ten weeks; about eleven weeks; about twelve weeks; about thirteen weeks, about fourteen weeks; about fifteen weeks; about sixteen weeks; about seventeen weeks; about eighteen weeks; about nineteen weeks; about twenty weeks; about twenty-one weeks; about twenty-two weeks; about twenty-three weeks; about twenty four weeks; about seven months; about eight months; about nine months; about ten months; about eleven months; about twelve months; about thirteen months; about fourteen months; about fifteen months; about sixteen months; about seventeen months; about eighteen months; about nineteen months; about twenty months; about twenty one months; about twenty-two months; about twenty-three months; about twenty-four months; about thirty months; about three years; about four years and about five years.

With regard to the frequency of administering the innate immunity modifier (e.g., talabostat mesylate), one of ordinary skill in the art will be able to determine an appropriate frequency. For example, a clinician can decide to administer the talabostat mesylate (once a daily, once in two day, once in three days, once in four days, once in five days, once in six days, once a week, once in two weeks, once in three weeks, once a month). In certain embodiments, the innate immunity modifier (for example talabostat mesylate) is administered three doses per day, two doses per day, one dose per day, one dose every 2 days, one dose every 3 days, one dose every 4 days, one dose every 5 days, once a week, once every two weeks, once every three weeks or once every four weeks, preferably once a day.

With regard to the frequency of administering the immune checkpoint inhibitor (e.g., PD-1 axis antagonist such as an antibody against PD-1), one of ordinary skill in the art will be able to determine an appropriate frequency. For example, a clinician can decide to administer the PD-1 axis antagonist (e.g. once every three weeks, once every two weeks or once a week). In certain embodiments, PD-1 antagonist is administered one dose per day or one dose every 2 days or one dose every 3 days or one dose every 4 days or one dose every 5 days or once a week (Q1W), once every two weeks (Q2W) or once every three weeks (Q3W) or once every four weeks (Q4W), twice a week or twice every two weeks or twice every three weeks or twice every four weeks, preferably twice every four weeks. In certain embodiments, the PD-1 antagonist is administered as a single dose, in two doses, in three doses, in four doses, in five doses, or in 6 or more doses. The dosing schedule can vary from e.g., once a week to once every 2, 3, 4 weeks or twice a week to twice every 2, 3, or 4 weeks.

With regard to the frequency of administering the T-cell stimulator (for example a an IL2Rβ selective agonist such as a PEGylated IL-2), one of ordinary skill in the art will be able to determine an appropriate frequency. For example, a clinician can decide to administer the T-cell stimulator relatively infrequently (e.g., once every eight weeks (Q8W), or once every seven weeks (Q7W), or once every six weeks (Q6W), or once every five weeks (Q5W), or once every four weeks (Q4W), or once every three weeks (Q3W), or once every two weeks (Q2W) or once every 9 days (Q9D)) as deemed appropriate. In some embodiments, the T-cell stimulator is administered once every three weeks (Q3W). In addition, as some innate immunity modifiers, immune checkpoint inhibitors and T-cell stimulators, are either in advanced clinical testing or commercially available, it is also possible to refer to the literature to obtain an appropriate frequency of administration (keeping in mind that some adjustment may be necessary in view of the combined effects of the treatment regimen).

In some embodiments, an innate immunity modifier, can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week or 2 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week or 2 weeks after) the administration of a PD-1 axis antagonist or a T-cell stimulator (for example PEGylated IL-2 such as RSLAIL-2), to a subject with cancer. In certain embodiments, one agent may be administered more frequently than the other agent(s) such that multiple doses of one agent are administered for each dose of the other agent(s).

In other embodiment, administration of a selective dipeptidyl peptidase inhibitor, an immune checkpoint inhibitor, and an IL2RI selective agonist such as a PEGylated IL-2, whether simultaneous, sequential (in any order) or both, can be performed according to any number of desired intervals of minutes (e.g., 0-60 minutes), hours (e.g., 0-24 hours), days (e.g., 0-7 days), and/or weeks (e.g., 0-52 weeks) as can be decided and determined by one of skill in the art. Exemplary dosages and dosing intervals can also vary over time (e.g., depending upon the patient's clinical response, side effects, etc.), or during different phases of therapy (induction, treatment, or maintenance).

Assays for determining whether a given compound can act as an innate immune modifier can be determined through routing experimentation by one of ordinary skill in the art.

In accordance with the method described herein, the innate immunity modifier preferably selective dipeptidyl peptidase inhibitor is administered to a patient in a dipeptidyl peptidase inhibiting amount. One of ordinary skill in the art can determine how much a given a selective dipeptidyl peptidase inhibitor sufficient to provide clinically relevant inhibitory activity at DPP8/9/FAP.

In another embodiment, the dosage of the selective dipeptidyl peptidase inhibitor administered to prevent and/or treat a cancer associated with increased levels of FAP or DPP 8/9 in a patient includes about 0.001 mg/kg to about 10 mg/kg, about 0.001 mg/kg to about 1 mg/kg, about 0.001 mg/kg to 0.5 mg/kg, about 0.001 mg/kg to 0.2 mg/kg, 0.001 mg/kg to about 0.1 mg/kg, about 0.001 mg/kg to 0.05 mg/kg, about 0.001 mg/kg to 0.035 mg/kg, about 0.002 mg/kg to about 1 mg/kg, about 0.002 mg/kg to about 0.5 mg/kg, about 0.002 mg/kg to about 0.2 mg/kg, about 0.002 mg/kg to about 0.1 mg/kg, about 0.002 mg/kg to about 0.05 mg/kg, about 0.002 mg/kg to about 0.035 mg/kg, about 0.003 mg/kg to about 1 mg/kg, 0.003 mg/kg to about 0.5 mg/kg, 0.003 mg/kg to about 0.2 mg/kg, about 0.004 mg/kg to about 0.1 mg/kg, about 0.005 mg/kg to about 0.05 mg/kg, about 0.006 mg/kg to about 0.05 mg/kg, about 0.007 mg/kg to about 0.05 mg/kg, about 0.008 mg/kg to about 0.05 mg/kg, about 0.009 mg/kg to about 0.05 mg/kg, about 0.010 mg/kg to about 0.05 mg/kg, about 0.011 mg/kg to about 0.05 mg/kg, about 0.012 mg/kg to about 0.05 mg/kg, about 0.013 mg/kg to about 1 mg/kg, The dose of a selective dipeptidyl peptidase inhibitor may vary from about 0.001 mg/kg to 2 mg/kg, about 0.001 mg/kg to 1 mg/kg, preferably 0.001 mg/kg to 0.5 mg/kg, more preferably about 0.001 mg/kg to 0.2 mg/kg. Total daily dose of a selective dipeptidyl peptidase inhibitor may vary from about 100 mcg to 200 mg, preferably about 100 mcg to 50 mg, most preferably about 100 mcg to 10 mg.

In certain embodiments, the innate immunity modifier (for example talabostat mesylate) is administered in a dose of 0.001 mg/kg, 0.002 mg/kg, 0.003 mg/kg, 0.004 mg/kg, 0.005 mg/kg, 0.006 mg/kg, 0.007 mg/kg, 0.008 mg/kg, 0.009 mg/kg, 0.010 mg/kg, 0.011 mg/kg, 0.012 mg/kg, 0.013 mg/kg, 0.014 mg/kg, 0.015 mg/kg, 0.016 mg/kg, 0.017 mg/kg, 0.018 mg/kg, 0.019 mg/kg, 0.020 mg/kg, 0.025 mg/kg, 0.030 mg/kg, 0.035 mg/kg, 0.06 mg/kg and 0.08 mg/kg. In preferred embodiments, each dose of the selective dipeptidyl peptidase inhibitor is administered at 0.002 mg/kg, 0.003 mg/kg, 0.004 mg/kg, 0.005 mg/kg, 0.006 mg/kg, 0.007 mg/kg, 0.009 mg/kg, 0.01 mg/kg, 0.013 mg/kg and 0.014 mg/kg. The dose of talabostat or a pharmaceutically acceptable salt thereof may vary from about 0.001 mg/kg to 0.0.024 mg/kg mg/kg, preferably 0.001 mg/kg to 0.017 mg/kg, preferably 0.001 mg/kg to 0.014 mg/kg, more preferably about 0.001 mg/kg to 0.010 mg/kg and more preferably about 0.001 mg/kg to 0.009 mg/kg. Total daily dose of talabostat mesylate may vary from about 50 micrograms to 2 mg, preferably about 100 micrograms to 1.2 mg, more preferably about 100 micrograms to 1.2 mg, most preferably 100 micrograms to 600 micrograms

In some embodiments, talabostat mesylate is administered at a daily dose of about 100 micrograms to about 600 micrograms during the treatment phase in a dose escalation manner, as required. In some embodiments, talabostat mesylate is formulated for oral administration.

In accordance with the method described herein, an immune checkpoint inhibitor includes PD-1 axis antagonist, CTLA4 antagonist and combination thereof. In accordance with the method described herein, a CTLA-4 pathway-inhibiting amount of an anti-CTLA-4 antibody is administered or a PD-1 pathway-inhibiting amount of an anti-PD-1 antibody is administered. One of ordinary skill in the art can determine how much a given anti-CTLA-4 antibody or anti-PD-1 antibody is sufficient to provide clinically relevant inhibition of the CTLA-4 pathway or PD-1 pathway, respectively. For example, one of ordinary skill in the art can refer to the literature and/or administer a series of increasing amounts the anti-CTLA-4 antibody or anti-PD-1 antibody and determine which amount or amounts provide clinically relevant inhibition the CTLA-4 pathway or PD-1 pathway. In one or more instances, the PD-1 axis antagonist amounts are encompassed by one or more of the following ranges (encompassing human doses): from about 0.1 mg/kg to about 10 mg/kg; from about 1 mg/kg to about 9 mg/kg; from about 0.5 mg/kg to about 8 mg/kg; from about 0.5 mg/kg to about 7 mg/kg; from about 0.5 mg/kg to about 6 mg/kg; from about 0.5 mg/kg to about 5 mg/kg; from about 0.5 mg/kg to about 4 mg/kg; from about 0.5 mg/kg to about 3 mg/kg; from about 0.5 mg/kg to about 2 mg/kg; from about 0.5 mg/kg to about 2 mg/kg.

In one or more instances, the CTLA4 antagonist amounts are encompassed by one or more of the following ranges (encompassing human doses): from about 0.1 mg/kg to about 10 mg/kg; from about 0.5 mg/kg to about 10 mg/kg; from about 1 mg/kg to about 10 mg/kg; from about 1.5 mg/kg to about 10 mg/kg; from about 2 mg/kg to about 10 mg/kg; from about 3 mg/kg to about 10 mg/kg.

For confirmation, as used herein with regard to CTLA-4 and PD-1 axis antagonist amounts of the CTLA-4 antagonist and PD-1 axis antagonist respectively, the amount and extent of the inhibition can vary widely and the combination of either of these with the innate immunity modifier and an IL-2RO-selective agonist such as RSLAIL-2) can still be effective. For example, an amount of the CTLA-4 antagonist or PD-1 antagonist that only minimally inhibits the CTLA-4 or PD-1 pathways, respectively, can still be an inhibiting amount as used herein so long as the method results in a clinically meaningful response.

In one or more preferred embodiments, the PD-1 axis antagonist in the combination therapy is a PD-1 antagonist such as nivolumab, which is administered intravenously at a dose selected from: 0.5 mg/kg Q2W, 1 mg/kg Q2W, 240 mg Q2W, 2 mg/kg Q2W, 3 mg/kg Q2W, 5 mg/kg Q2W, 10 mg/kg Q2W, 1 mg/kg Q3W, 2 mg/kg Q3W, 3 mg/kg Q3W, 5 mg/kg Q3W, and 10 mg/kg Q3W and flat-dose equivalents of any of these doses, such as 240 mg Q2W. The preferred doses are about 5 mg/kg Q2W, about 1 mg/kg Q2W, about 240 mg Q2W, about 2 mg/kg Q2W and about 3 mg/kg Q2W.

In another preferred embodiment, the PD-1 axis antagonist in the combination therapy is a PD-1 antagonist such as MK-3475, which is administered in a liquid medicament at a dose selected from 1 mg/kg Q2W, 2 mg/kg Q2W, 3 mg/kg Q2W, 5 mg/kg Q2W, 10 mg/kg Q2W, 1/kg Q3W, 2 mg/kg Q3W, 3 mg/kg Q3W, 5 mg/kg Q3W, 10 mg/kg Q3W and flat-dose equivalents of any of these doses, such as 200 mg Q3W, preferably about 2 mg/kg Q2W, about 200 mg Q3W and combination thereof. In some particularly preferred embodiments, MK-3475 is administered as a liquid medicament which comprises 25 mg/ml MK-3475, 7% (w/v) sucrose, 0.02% (w/v) polysorbate 80 in 10 mM histidine buffer pH 5.5, and the selected dose of the medicament is administered by IV infusion over a time period of about 30 minutes.

In some embodiments, a pharmaceutical composition comprising an anti-PD-1 antibody as the PD-1 antagonist may be provided as a liquid formulation or prepared by reconstituting a lyophilized powder with sterile water for injection prior to use. PCT Publication Appl. No. WO2012/135408 describes the preparation of liquid and lyophilized medicaments comprising pembrolizumab that are suitable for use. In some embodiments, a medicament comprising pembrolizumab is provided in a glass vial which contains about 100 mg of pembrolizumab in 4 ml of solution. Each 1 mL of solution contains 25 mg of pembrolizumab and is formulated in: L-histidine (1.55 mg), polysorbate 80 (0.2 mg), sucrose (70 mg), and Water for Injection, USP. The solution requires dilution for IV infusion.

In one or more preferred embodiments, the CTLA4 antagonist in the combination therapy is a CTLA4 antagonist such as ipilimumab, which is administered intravenously at a dose selected from: 3 mg/kg Q3W for 4 doses, followed by 10 mg/kg every 12 weeks for up to 3 weeks or until the documented disease recurrence or unacceptable toxicity.

In accordance with the method described herein, the IL-2 receptor agonist is administered to a patient in an IL-2-activating amount. One of ordinary skill in the art can determine how much a given long acting, IL-2-selective agonist sufficient to provide clinically relevant agonistic activity at IL-2. For example, one of ordinary skill in the art can refer to the literature and/or administer a series of increasing amounts the long acting, IL-2 agonist and determine which amount or amounts provide clinically agonistic activity of IL-2.

In one or more instances, the T-cell stimulator, for example an IL2Rβ biased agonist e.g. RSLAIL-2) is used in an amount encompassed by one or more of the following ranges (encompassing human doses): from about 0.001 mg/kg to about 10 mg/kg; about 0.001 mg/kg to about 5 mg/kg, about 0.001 mg/kg to about 4 mg/kg, about 0.001 mg/kg to about 3 mg/kg, about 0.001 mg/kg to about 2 mg/kg, about 0.001 mg/kg to about 1 mg/kg, about 0.001 mg/kg to about 0.01 mg/kg or about 0.001 mg/kg to about 0.1 mg/kg.

In yet other certain embodiments, the amount of the T-cell stimulator, e.g., multi(2,7-(bis-methoxyPEG-carboxyamide)(9H-fluorene-9-yl)methyl N-carbamate)interleukin-2, is used in the compositions and methods provided herein, is from about 0.0005 to about 0.3 mg/kg; from about 0.001 mg/kg to about 0.3 mg/kg; from about 0.001 mg/kg to about 0.25 mg/kg; from about 0.001 mg/kg to about 0.15 mg/kg; from about 0.001 mg/kg to about 0.05 mg/kg; from about 0.001 mg/kg to about 0.01 mg/kg; from about 0.001 mg/kg to about 0.008 mg/kg; from about 0.001 mg/kg to about 0.005 mg/kg; from about 0.002 mg/kg to about 0.005 mg/kg; and from about 0.002 mg/kg to about 0.004 mg/kg.

In some embodiments, multi(2,7-(bis-methoxyPEG-carboxyamide)(9H-fluorene-9-yl)methyl N-carbamate)interleukin-2 is administered at a dose that is less than or equal to 0.003 mg/kg. In certain embodiments, the dosing ranges include, for example, from about 0.001 mg/kg to about 0.01 mg/kg, or from about 0.002 mg/kg to about 0.008 mg/kg or from about 0.002 mg/kg to less than about 0.006 mg/kg. In certain embodiments, multi(2,7-(bis-methoxyPEG-carboxyamide)(9H-fluorene-9-yl)methyl N-carbamate)interleukin-2 used in the compositions and methods provided herein, is administered once every 3 weeks. Dosages for multi(2,7-(bis-methoxyPEG-carboxyamide)(9H-fluorene-9-yl)methyl N-carbamate)interleukin-2 are based upon IL-2 equivalents unless otherwise indicated.

In a particular embodiment directed to a triple combination, the innate immunity modifier (for example, talabostat or a pharmaceutically acceptable salt thereof) is orally administered once a day at a dose range of about 100 micrograms to about 600 micrograms during a 21-day cycle simultaneously with an every three-week (Q3W) dose schedule of PD-1 antagonist at a dose from about 0.5 mg/kg to about 2 mg/kg and an every three week (Q3W) dose schedule of multi(2,7-(bis-methoxyPEG-carboxyamide)(9H-fluorene-9-yl)methyl N-carbamate)interleukin-2 at a dose range of about 0.003 mg/kg to about 0.006 mg/kg, where the administration cycles are repeated with an appropriate rest period as per the disease progression, ideally until complete disease resolution is achieved or until any significant toxicity is observed.

The optimal dose for a combination of talabostat mesylate, nivolumab and multi(2,7-(bis-methoxyPEG-carboxyamide)(9H-fluorene-9-yl)methyl N-carbamate)interleukin-2 may be identified by dose escalation or dose de-escalation of one or more of these agents.

In an embodiment, the combination therapy comprises a 21-day treatment cycle in which talabostat mesylate is orally administered once a day from about 100 micrograms to 600 micrograms, nivolumab is parenterally administered at about 0.5 mg/kg to 1.5 mg/kg Q2W and multi(2,7-(bis-methoxyPEG-carboxyamide)(9H-fluorene-9-yl)methyl N-carbamate)interleukin-2, is parenterally administered at a dose range of about 0.003 mg/kg to 0.006 mg/kg Q3W.

In an embodiment, the combination therapy comprises a 21-day treatment cycle in which talabostat mesylate is orally administered from about 100 micrograms to 600 micrograms, pembrolizumab is parenterally administered at 200 mg Q3W and multi(2,7-(bis-methoxyPEG-carboxyamide)(9H-fluorene-9-yl)methyl N-carbamate)interleukin-2 is parenterally administered at 0.006 mg/kg Q3W.

In particular aspects, the optimal dose for a combination of talabostat mesylate, pembrolizumab and multi(2,7-(bis-methoxyPEG-carboxyamide)(9H-fluorene-9-yl)methyl N-carbamate)interleukin-2, may be identified by dose escalation or dose de-escalation of one or more of these agents. In some particular embodiments, the administration is oral or parenteral or both.

The treatment method as described herein can continue for as long as the clinician overseeing the patient's care deems the treatment method is effective. Non-limiting parameters that indicate the treatment method is effective include the following: tumor shrinkage (in terms of weight and/or volume); a decrease in the number of individual tumor colonies; tumor elimination; and/or progression-free survival.

The efficacy of the treatment methods provided herein can be assessed using any suitable means. In one embodiment, the treatment produces at least one therapeutic effect selected from the group consisting of reduction in size of a tumor, reduction in number of metastatic lesions overtime, complete response, partial response, and stable disease. In another embodiment, administration of an innate immune modifier, an immune checkpoint inhibitor, and a T-cell stimulator results in at least a 1-, 1.25-, 1.50-, 1.75-, 2-, 2.25-, 2.50-, 2.75-, 3-, 3.25-, 3.5-, 3.75-, or 4-fold reduction in tumor volume, e.g., relative to treatment with the innate immune modifier or the immune checkpoint inhibitor or the T-cell stimulator alone, or relative to tumor volume before initiation of the treatment.

In particular aspects, administration of an innate immune modifier, an immune checkpoint inhibitor, and a T-cell stimulator results in tumor growth inhibition of at least 50%, 60%, 70%, 80%, 90%, 100% e.g., relative to treatment with the innate immune modifier or the immune checkpoint inhibitor or the T-cell stimulator alone, or relative to tumor volume before initiation of the treatment. In certain embodiments, tumor volume is reduced by at least 50%, 60%, 70%, 80%, 90% or more, e.g., relative to tumor size before initiation of the treatment.

### V: INDICATIONS FOR TREATMENT

In some embodiments, we provide herein a method of treating cancer in a subject, comprising administering to the subject an effective amount of an innate immunity modifier (for example a selective dipeptidyl peptidase inhibitor), an effective amount of an immune checkpoint inhibitor and an effective amount of a T-cell stimulator (for example PEGylated IL-2).

Patient suffering from a condition that is responsive to treatment with one of the individual therapeutic agents of the combination of the present invention may be treated with the combination of the present invention. For example, patients may be responsive to the individual agents alone as well as the combination, but exhibit a greater response to the combination. By way of further example, patients may be non-responsive to one of the individual agents, but are responsive to a combination of two agents (e.g. a selective dipeptidyl peptidase inhibitor and a T-cell stimulator) and yet more responsive to all three agents (e.g. a selective dipeptidyl peptidase inhibitor, a T-cell stimulator and an immune checkpoint inhibitor).

In some embodiments, we provide herein methods and compositions for inducing or enhancing an immune response in a host for the treatment cancer. Because these methods operate by blocking inhibitory receptors present on T-cells and NK cells, they are applicable to a very broad range of cancers.

Any of the provided methods can be used to treat a cancer that is a tumor, such as a solid tumor. In particular aspects, the tumor is characterized as having a moderate to high dipeptidyl peptidase expression, specifically FAP expression or DPP 8/9 expression. Exemplary cancers that can be treated by the provided methods include, but are not limited to, pancreatic cancer, colorectal cancer, prostate cancer, ovarian cancer, neuroendocrine prostate cancer (NePC), (e.g., treatment induced neuroendocrine prostate cancer (tnepc)), hormone refractory prostate cancer, castration resistant prostate cancer (CrPC), lung cancer, breast cancer, glioblastoma, gastric cancer, astroglial cancer, neuroectodermal tumors, head and neck cancer, triple negative breast cancer, gastroesophageal cancer and non-small cell lung cancer. The present combination, compositions, and related methods are also useful for the treatment of metastatic cancers, especially metastatic cancers that express PDL-1 or CTLA4.

Particular cancers whose growth may be inhibited using the combination therapy comprising a selective dipeptidyl peptidase inhibitor (for example, talabostat or a pharmaceutically acceptable salt thereof), an immune checkpoint inhibitor and an IL2Rβ biased agonist, such as a PEGylated IL-2, for example, RSLAIL-2) include cancers typically responsive to immunotherapy.

In some embodiments, the cancer/tumor is a urogenital cancer (such as prostate cancer, treatment induced neuroendocrine prostate cancer, hormone sensitive or hormone refractory prostate cancer, castration resistant prostate cancer, renal cell cancer, bladder cancer), renal cancer (e.g., clear cell carcinoma), thyroid cancer, testicular cancer, vulvar cancer, Wilms tumor, gynecological cancers (such as ovarian cancer, cervical cancer, endometrial cancer, uterine cancer), lung cancer, non-small cell lung cancer, small cell lung cancer, gastrointestinal stromal cancer, gastrointestinal cancers (such as non-metastatic or metastatic colorectal cancer, pancreatic cancer, gastric cancer, oesophageal cancer, hepatocellular cancer, cholangiocellular cancer), malignant glioblastoma, malignant mesothelioma, non-metastatic or metastatic breast cancer (such as hormone refractory metastatic breast cancer, triple negative breast cancer), liver cancer, malignant melanoma, melanoma, metastatic melanoma, merkel cell carcinoma or bone and soft tissue sarcomas, squamous cell cancer (e.g. oral squamous cell carcinoma), squamous and non-squamous lung cancer, glioblastoma, brain cancer, osteosarcoma, neuroblastoma, advanced metastatic, neuroectodermal tumors, an inflammatory myofibroblastic tumor (IMT), cholangiocarcinoma, cystadenocarcionoma, diffuse large B cell lymphoma, myelodysplastic syndromes, adrenal cancer, uveal melanoma, hodgkin's disease, hepatocellular carcinoma, ameloblastoma, chondrosarcoma, dermatofibrosarcoma, ganglioglioma, leiomyosarcoma, medulloblastomma, osteoblastoma and inoperable non-inflammatory locally advanced disease, colon carcinoma, basal cell cancer, adenocarcinoma, sweat gland cancer, sebaceous gland cancer, papillary cancer, papillary adenocarcinomas, cystadenocarcinoma, medullary cancer, bronchogenic cancer, hepatoma, bile duct cancer, choriocarcinoma, seminoma, embryonal cancer, epithelial cancer, astrocytoma, medulloblastoma, craniopharyngioma, ependymoma, pinealoma, hemangioblastoma, acoustic neuroma, oligodendroglioma, meningioma, retinoblastoma, gastroesophageal, fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, hematopoietic cancer (leukemia, lymphoma, a lymphocytic leukemia, non-Hodgkin's lymphoma, Hodgkin's lymphoma, an anaplastic large-cell lymphoma, anaplastic astrocytoma, myeloid leukemia, multiple myeloma, acute lymphoblastic leukemia, chronic myeloid leukemia, acute myeloid leukemia), B cell lymphoma, and the like.

In a particular embodiment, the cancer is a solid tumor (such as pancreatic cancer, colorectal cancer, ovarian cancer, lung cancer, breast cancer, liver cancer, fibrosarcoma, glioblastoma, prostate cancer, hormone refractory prostate cancer, treatment induced neuroendocrine prostate cancer, castration resistant prostate cancer, malignant melanoma, thyroid cancer, gastric cancer, astroglial, neuroectodermal tumors, head and neck cancer, kidney cancer, cancer of the bile duct, brain cancer, cervical cancer, maxillary sinus cancer, bladder cancer, esophageal cancer, adrenocortical cancer, triple negative breast cancer, gastroesophageal cancer, non-small cell lung cancer, small cell lung cancer and the like) or hematopoietic cancer (such as leukemia, lymphoma, a lymphocytic leukemia, non-hodgkin's lymphoma, hodgkin's lymphoma, an anaplastic large-cell lymphoma, myeloid leukemia, multiple myeloma, acute lymphoblastic leukemia, chronic myeloid leukemia, acute myeloid leukemia and the like). Particular cancers of interest include pancreatic cancer and colorectal cancer.

In some embodiments, the cancers whose growth may be inhibited using combination therapy comprising at least one selective dipeptidyl peptidase inhibitor, at least one immune checkpoint inhibitor and at least one T-cell stimulator (for example PEGylated IL 2) are virally-associated cancers. Exemplary virally-associated cancers include, but are not limited to, cancers associated with Epstein-Barr virus (EBV), hepatitis B virus (HBV), hepatitis C virus (HCV), human papilloma viruses (HPV), human T lymphotropic virus type 1 (HTLV-1), human T lymphotropic type 2 (HTLV-2) and human herpesvirus, such as human herpesvirus 8 (HHV-8). The cancers associated with particular viruses are known to those of ordinary skill in the art. For example, examples of EBV-associated cancers include, but are not limited to, lymphomas, nasopharyngeal cancer, gastric carcinoma, parotid carcinoma, breast carcinoma, and leiomyosarcoma. Examples of cancers associated with hepatitis B virus (HBV) and hepatitis C virus (HCV) include but are not limited to cancers of the liver. Examples of cancers associated with human papilloma viruses (HPV) include, but are not limited to, oropharyngeal head and neck cancer, nasopharyngeal head and neck cancer, and cancers of the cervix, vulva, vagina, penis and anus. Examples of cancers associated with human T lymphotropic virus type 1 (HTLV-1) and type 2 (HTLV-2) include, but are not limited to, adult T-cell leukemia and hairy-cell leukemia, respectively. Examples of cancers associated with human herpesvirus 8 (HHV-8) include, but are not limited to, Kaposi sarcoma. In some embodiments, the virally-associated cancer is a cancer associated with HPV. In other embodiments, the virally-associated cancer is a cancer associated with HCV. In some embodiments, the subject is suffering from rare non-immunogenic cancer include but not limited to medulloepithelioma, alveolar soft tissue sarcoma, pleural mesothelioma, retinoblastoma, rhabdomyosarcoma, squamous cell carcinoma of head and neck, thymic carcinoma, thymoma, undifferentiated pleomorphic sarcoma, vaginal carcinoma or the like.

In some embodiments, the subject is a human. In some embodiments, the subject has cancer or has been diagnosed with cancer. In some embodiments, the subject is suffering from relapsed or refractory cancer (such as solid tumor). In some embodiments, the subject is suffering from pancreatic cancer, colorectal cancer, prostate cancer, castration resistant prostate cancer.

The methods disclosed herein may find use in treating conditions where enhanced immunogenicity is desired such as increasing tumor immunogenicity for the treatment of cancer. A variety of cancers may be treated, or their progression may be delayed, including but are not limited to, a cancer that is a solid tumor. In some embodiments, the cancer is a refractory or metastatic cancer. In some embodiments, the cancer is a lymphoma or a leukemia. In some embodiments, the leukemia is chronic lymphocytic leukemia (CLL) or acute myeloid leukemia (AML). In some embodiments, the lymphoma is follicular lymphoma (FL), diffuse large B-cell lymphoma (DLBCL), or non-hodgkin's lymphoma (NHL).

In particular aspects, tumors with high macrophage densities are particularly good candidates for the combination therapy. Macrophage density may be measured by immunohistochemistry or by flow cytometry. As used herein, high macrophage density measured by flow cytometry of the is at least 20%, at least 30% or at least 40% macrophages, relative to CD45-positive cells.

### VI: PHARMACEUTICAL COMPOSITIONS

Each therapeutic agent, namely an innate immunity modifier (such as talabostat or a pharmaceutically acceptable salt thereof), an immune checkpoint inhibitor (such as a PD-1 axis antagonist) and a T-cell stimulator (such as an IL2RP-specific agonist, optionally a PEGylated IL-2, e.g. RSLAIL-2) in a combination therapy as provided for herein may be administered as is, or in a pharmaceutical composition which comprises the therapeutic agent and one or more pharmaceutically acceptable carriers, excipients and diluents, according to standard pharmaceutical practice.

Each therapeutic agent may be formulated separately, and all the agents may be administered either at the same time or separately. Further, the three formulations may be placed in a single package, to provide the so-called kit formulation. In some configurations, all the compounds may be contained in a single formulation.

In another embodiment, provided herein is a pharmaceutical composition to treat a cancer in a subject, comprising: a therapeutically effective amount of an innate immunity modifier (for example talabostat or a pharmaceutically acceptable salt thereof), an immune checkpoint inhibitor (for example a PD-1 axis antagonist) and T-cell stimulator (for example, an IL2RP-specific agonist, optionally a PEGylated IL-2, e.g. RSLAIL-2). In some embodiments, (a) a first pharmaceutical composition comprises talabostat or a pharmaceutically acceptable salt thereof together with one or more pharmaceutically acceptable carriers and/or excipients, (b) a second pharmaceutical composition comprises a PD-1 axis antagonist with one or more pharmaceutically acceptable carriers and/or excipients, and (c) a third pharmaceutical composition comprises an IL2RP-specific agonist, optionally a PEGylated IL-2, e.g. RSLAIL-2, together with one or more pharmaceutically acceptable carriers and/or excipients. The compositions may be administered to the subject at the same time, sequentially in any suitable order or separately (including intermittently), such that the combination therapy provides an effective treatment of cancer in said subject.

In other aspects, the present disclosure provides two separate pharmaceutical compositions, namely (1) a pharmaceutical composition comprising an innate immunity modifier and an immune checkpoint inhibitor together with one or more pharmaceutically acceptable carriers and/or excipients and (2) a pharmaceutical composition comprising T cell stimulator together with one or more pharmaceutically acceptable carriers and/or excipients, or (1) a pharmaceutical composition comprising an innate immunity modifier together with one or more pharmaceutically acceptable carriers and/or excipients and (2) a pharmaceutical composition comprising an immune checkpoint inhibitor and a T cell stimulator together with one or more pharmaceutically acceptable carriers and/or excipients, or (1) a pharmaceutical composition comprising an innate immunity modifier and a T cell stimulator together with one or more pharmaceutically acceptable carriers and/or excipients and (2) a pharmaceutical composition comprising an immune checkpoint inhibitor together with one or more pharmaceutically acceptable carriers and/or excipients. The compositions may be administered to the subject at the same time, sequentially in any suitable order or separately (including intermittently), such that the combination therapy provides an effective treatment of cancer in said subject.

In one embodiment, the innate immunity modifier is a selective dipeptidyl peptidase inhibitor, said selective dipeptidyl peptidase inhibitor is preferably a small molecule.

In another embodiment, the immune checkpoint inhibitor is a PD-1 axis antagonist.

In a further embodiment, the immune checkpoint inhibitor is a CTLA4 antagonist.

In yet another embodiment, the T cell stimulator comprises an IL2RP-specific agonist, optionally a PEGylated IL-2, such RSLAIL-2.

In a preferred embodiment, the selective dipeptidyl peptidase inhibitor is talabostat or a pharmaceutically acceptable salt thereof, e.g. talabostat mesylate.

In a further preferred embodiment, the immune checkpoint inhibitor is a PD-1 axis antagonist, e.g. a PD-1 antagonist (for example an anti-PD-1 antibody), a PDL-1 antagonist (for example an anti-PDL-1 antibody) or a PDL-2 antagonist (for example an anti-PDL-2 antibody).

In another preferred embodiment, the immune checkpoint inhibitor is a CTLA4 antagonist.

In preferred embodiments, all the therapeutic agents are administered via separate pharmaceutical formulations.

In another embodiment, the separate pharmaceutical formulations are placed in a single package, to provide a so-called “kit formulation”.

In a particular embodiment, a pharmaceutical composition comprises talabostat or a pharmaceutically acceptable salt thereof (e.g. talabostat mesylate) in the form of an oral tablet.

In another particular embodiment, a pharmaceutical composition comprises a PD-1 axis antagonist in the form of a parenteral formulation.

In a further embodiment, a pharmaceutical composition comprises a PEGylated IL-2 in the form of a parenteral formulation.

Therapeutically effective amounts of the active agents may conveniently be administered via injection or oral. Other modes of administration are also contemplated, such as pulmonary, nasal, buccal, rectal, sublingual, enteral and transdermal. As used herein, the term “parenteral” includes subcutaneous, intravenous, intra-arterial, intraperitoneal, intracardiac, intrathecal, and intramuscular injection, as well as infusion injections. Each active component can be administered separately. Alternatively, if administration of two active components (e.g. a T-cell stimulator and an immune checkpoint inhibitor) is desired to be simultaneous and the two active components are compatible together in a given formulation then the simultaneous administration can be achieved via administration of single dosage form/formulation (e.g., intravenous administration of an intravenous formulation that contains the pharmacologically active agents). One of ordinary skill in the art can determine through routine testing whether two given pharmacological components are compatible together in a given formulation.

The pharmaceutical compositions may be formulated in a variety of ways, including for example, liquid, semi-solid and solid dosage forms, such as liquid solutions (e.g., injectable and infusible solutions), dispersions or suspensions, tablets, pills, powders, liposomes and suppositories. In some embodiments, the compositions may be formulated as the injectable or infusible solutions. The composition is in a form suitable for oral, intravenous, intraarterial, intramuscular, subcutaneous, parenteral, transmucosal, transdermal, or topical administration. The composition may be formulated as an immediate, controlled, extended or delayed release composition.

In some embodiments, the composition of the invention (e.g., talabostat or a pharmaceutically acceptable salt thereof) may be administered orally. In other embodiments, the composition of the invention (e.g. a PD-1 axis antagonist) may be administered by intravenous, intramuscular or subcutaneous injection. In yet other embodiments, the composition of the invention (e.g., a T-cell stimulator) may be administered parenterally (e.g., intravenous, subcutaneous, intraperitoneal, intramuscular).

The pharmaceutical composition of the present invention may also contain one or more pharmaceutically acceptable carriers or excipients.

Pharmaceutically acceptable carriers include water; saline; phosphate buffered saline; dextrose; glycerol; alcohols such as ethanol and isopropanol; phosphate, citrate and other organic acids; ascorbic acid; low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; EDTA; salt forming counterions such as sodium; and/or nonionic surfactants such as TWEEN, polyethylene glycol (PEG), and PLURONICS; isotonic agents such as sugars, polyalcohols such as mannitol and sorbitol, and sodium chloride; as well as combinations thereof. Antibacterial and antifungal agents include parabens, chlorobutanol, phenol, ascorbic acid and thimerosal.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Other common parenteral vehicles include sodium phosphate solutions, Ringer's dextrose, dextrose and sodium chloride, lactated Ringer's, or fixed oils. Intravenous vehicles include fluid and nutrient replenishers, electrolyte replenishers, such as those based on Ringer's dextrose, and the like. Preservatives and other additives may also be present such as for example, antimicrobials, antioxidants, chelating agents, and inert gases or the like.

More particularly, pharmaceutical compositions suitable for injectable use include sterile aqueous solutions (where water soluble) or dispersions and sterile powders for the extemporaneous preparation of sterile injectable solutions or dispersions. In such cases, the composition must be sterile and should be fluid to the extent that easy syringability exists. It should be stable under the conditions of manufacture and storage and will preferably be preserved against the contaminating action of microorganisms, such as bacteria and fungi. The carrier can be a solvent or dispersion medium containing, for example, water, ethanol, polyol (e.g., glycerol, propylene glycol, liquid polyethylene glycol, or the like), and suitable mixtures thereof. The proper fluidity can be maintained, for example, by the use of a coating such as lecithin, by the maintenance of the required particle size in the case of dispersion and by the use of surfactants. Suitable formulations for use in the therapeutic methods disclosed herein are described in Remington's Pharmaceutical Sciences, Mack Publishing Co., 16th ed. (1980).

In some embodiments, the composition includes isotonic agents, for example, sugars, polyalcohols, such as mannitol, sorbitol, or sodium chloride. Prolonged absorption of the injectable compositions can be brought about by including in the composition an agent which delays absorption, for example, aluminum monostearate and gelatin.

Sterile injectable solutions can be prepared by incorporating the molecule, by itself or in combination with other active agents, in the required amount in an appropriate solvent with one or a combination of ingredients enumerated herein, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, one method of preparation is vacuum drying and freeze-drying, which yields a powder of an active ingredient plus any additional desired ingredient from a previously sterile-filtered solution thereof. The preparations for injections are processed, filled into containers such as ampoules, bags, bottles, syringes or vials, and sealed under aseptic conditions according to methods known in the art. Such articles of manufacture will preferably have labels or package inserts indicating that the associated compositions are useful for treating a subject suffering from or predisposed to autoimmune or neoplastic disorders.

For oral use, the pharmaceutical compositions of the present invention may be administered, for example, in the form of tablets or capsules, powders, dispersible granules, or cachets, or as aqueous solutions or suspensions. Oral compositions generally include an inert carrier (for example, diluent) or an edible carrier. They can also be enclosed in gelatin capsules or compressed into tablets. For oral administration, the therapeutic agents can be combined with carriers and used in the form of tablets, troches, or capsules. Pharmaceutically compatible binding agents, and/or adjuvant materials can be included as part of the composition. The tablets, pills, capsules, troches, and the like can contain any of the following ingredients, or compounds of a similar nature; a binder such as microcrystalline cellulose, gum tragacanth or gelatin; an excipient such as starch or lactose, a disintegrating agent such as alginic acid, primogel, or corn starch; a lubricant such as magnesium stearate or stearates; a glidant such as colloidal silicon dioxide; a sweetening agent such as sucrose or saccharin; or a flavoring agent such as peppermint, methyl salicylate, or orange.

Various methods can be used for manufacturing tablets. More particularly, the process may include dissolving talabostat mesylate in a suitable solvent (with or without binder) and this solution is distributed uniformly all over filler particles (may contain other materials) to form agglomerated particles/granules. Wet granulation or coating or spraying process can also be used. Obtained granules are appropriately sized or the granules can be further processed by dry granulation/slugging/roller compaction method followed by milling step to achieve suitable granules of specific particle size distribution. The sized granules are further blended with other components and/or and then lubricated in a suitable blender and compressed into tablets of specific dimensions using appropriate tooling. The coating can be done with appropriate equipment.

Also provided herein is a kit comprising a therapeutically effective amount of an innate immunity modifier (such as talabostat mesylate), an immune checkpoint inhibitor (such as a PD-1 axis antagonist) and a T-cell stimulator (such as an IL2RP-specific agonist, optionally a PEGylated IL-2, e.g. RSLAIL-2).

In some embodiments, a combination includes a formulation of an innate immunity modifier (for example a selective dipeptidyl peptidase inhibitor), an immune checkpoint inhibitor and T-cell stimulator (such as an IL2Rβ-specific agonist, optionally a PEGylated IL-2, e.g. RSLAIL-2), with or without instructions for combined use or to combination products. The combined therapeutics can be manufactured and/or formulated by the same or different manufacturers. The combination therapeutics may thus be entirely separate pharmaceutical dosage forms or pharmaceutical compositions that are also sold independently of each other. In some embodiments, instructions for their combined use are provided: (i) prior to release to physicians (e.g. in the case of a “kit” comprising a first therapeutic agent, second therapeutic agent and the third therapeutic agent); (ii) by the physicians themselves (or under the guidance of a physician) shortly before administration; (iii) the patient themselves by a physician or medical staff.

In one example, a single bolus dose may be administered. In another example, several divided doses may be administered over time. In yet another example, a dose may be proportionally reduced or increased as indicated by the exigencies of the therapeutic situation. Dosage unit form, as used herein, refers to physically discrete units suited as unitary dosages for treating mammalian subjects. Each unit may contain a predetermined quantity of active compound calculated to produce a desired therapeutic effect. In some embodiments, the dosage unit forms of the invention are dictated by and directly dependent on the unique characteristics of the active compound and the particular therapeutic or prophylactic effect to be achieved.

These and other aspects of the invention, including the exemplary specific embodiments listed below, will be apparent from the teachings contained herein.

### VII: SPECIFIC EMBODIMENTS OF THE INVENTION

### Embodiment 1

A method of treating cancer (e.g. a solid tumor) in a cancer comprising administering to a subject at least one innate immune modifier, at least one immune checkpoint inhibitor, and at least one T-cell stimulator.

### Embodiment 2

The method of Embodiment 1 wherein the cancer is pancreatic cancer, colorectal cancer, prostate cancer, hormone refractory prostate cancer, treatment induced neuroendocrine prostate cancer, castration resistant prostate cancer, ovarian cancer, lung cancer, breast cancer, glioblastoma, gastric cancer, malignant melanoma, liver cancer, kidney cancer, cancer of the bile duct, cervical cancer, maxillary sinus cancer, bladder cancer, astroglial cancer, neuroectodermal tumors, adrenocortical cancer, head and neck cancer, triple negative breast cancer, gastroesophageal cancer, non-small cell lung cancer or the like.

### Embodiment 3

The method of Embodiment 1 wherein the cancer is pancreatic cancer.

### Embodiment 4

The method of Embodiment 1 wherein the innate immune modifier is a selective dipeptidyl peptidase inhibitor.

### Embodiment 5

The method of Embodiment 5, wherein said selective dipeptidyl peptidase inhibitor is talabostat or a prodrug, analog, stereoisomer or related compound thereof, or a pharmaceutically acceptable salt of any of the foregoing, or a combination of such selective dipeptidyl peptidase inhibitors.

### Embodiment 6

The method of Embodiment 5, wherein said selective dipeptidyl peptidase inhibitor is talabostat or a pharmaceutically acceptable salt thereof.

### Embodiment 7

The method of Embodiment 6, wherein said talabostat or a pharmaceutically acceptable salt thereof is talabostat mesylate.

### Embodiment 8

The method of Embodiment 1 wherein the immune checkpoint inhibitor is a PD-1 axis antagonist or CTLA4 antagonist.

### Embodiment 9

The method of Embodiment 8, wherein the PD-1 axis antagonist is aPD-1 antagonist, a PD-L1 antagonist or a -PD-L2 antagonist.

### Embodiment 10

The method of Embodiment 9, wherein the PD-1 axis antagonist is a -PD-1 antagonist.

### Embodiment 11

The method of Embodiment 1 wherein the T-cell stimulator is an IL-2 receptor agonist.

### Embodiment 12

The method of Embodiment 11 wherein the IL-2 receptor agonist is interleukin-2 or a variant or derivative (e.g. prodrug) thereof.

### Embodiment 13

The method of Embodiment 11, wherein the interleukin-2 receptor agonist comprises multi(2,7-(bis-methoxyPEG-carboxyamide)(9H-fluorene-9-yl)methyl N-carbamate)interleukin-2.

### Embodiment 14

The method of Embodiment 13, wherein the multi(2,7-(bis-methoxyPEG-carboxyamide)(9H-fluorene-9-yl)methyl N-carbamate)interleukin-2 comprises (2,7-(bis-methoxyPEG_{10kD}-carboxyamide)(9H-fluorene-9-yl)methyl N-carbamate)_{6avg} interleukin-2 (“RSLAIL-2”).

### Embodiment 15

The method of Embodiment 9, wherein said PD-1 antagonist is selected from group consisting of ANA011, BGB-A317, KD033, pembrolizumab, MCLA-134, mDX400, MEDI0680, muDX400, nivolumab, PDR001, PF-06801591, pidilizumab, REGN-2810, SHR 1210, STI-Al110, TSR-042, ANB011, 244C8, 388D4, TSR042, BCD100, camrelizumab, JNJ63723283, JS001, spartalizumab, cemiplimab, tislelizumab, and XCE853, preferably pembrolizumab, or nivolumab.

### Embodiment 16

The method of Embodiment 9, wherein said PD-L1 antagonist is selected from group consisting of avelumab, BMS-936559, CA-170, durvalumab, MCLA-145, SP142, STI-A1011, STI-A1012, STI-A1010, STI-A1014, A110, KY1003, and atezolimumab, preferably avelumab.

### Embodiment 17

The method of Embodiment 9, wherein said PD-L2 antagonist is selected from AMP-224 and rHIgM12B7.

### Embodiment 18

The method of Embodiment 8, wherein said CTLA-4 antagonist is selected from the group consisting of KAHR-102, AGEN1884, ABR002, KN044, tremelimumab and ipilimumab, preferably tremelimumab or ipilimumab.

### Embodiment 19

A method of treating a cancer in a subject comprising administering to a subject talabostat mesylate, a PD-1 axis antagonist, and multi(2,7-(bis-methoxyPEG-carboxyamide)(9H-fluorene-9-yl)methyl N-carbamate)interleukin-2, which comprises RSLAIL-2.

### Embodiment 20

A method of Embodiment 19, wherein the cancer is pancreatic cancer, colorectal cancer, fibrosarcoma, colon cancer, colon adenocarcinoma or sarcoma, non-small cell lung cancer, prostate cancer, hormone refractory prostate cancer, treatment induced neuroendocrine prostate cancer, castration resistant prostate cancer, breast cancer, ovarian cancer, gastric cancer, malignant melanoma, head and neck cancer, liver cancer, small cell lung cancer, thyroid cancers, kidney cancer, cancer of the bile duct, brain cancer, cervical cancer, maxillary sinus cancer, bladder cancer, esophageal cancer, Hodgkin's disease, non-Hodgkin's lymphoma and adrenocortical cancer.

### Embodiment 21

The method of Embodiment 19, wherein the talabostat mesylate, the PD-1 axis antagonist, and multi(2,7-(bis-methoxyPEG-carboxyamide)(9H-fluorene-9-yl)methyl N-carbamate)interleukin-2, which comprises RSLAIL-2, are administered together as part of a single dosage form.

### Embodiment 22

The method of Embodiment 19, wherein the talabostat mesylate, the PD-1 axis antagonist, and multi(2,7-(bis-methoxyPEG-carboxyamide)(9H-fluorene-9-yl)methyl N-carbamate)interleukin-2, which comprises RSLAIL-2, are administered as separate individual dosage forms.

### Embodiment 22

A pharmaceutical combination for the treatment of cancer comprising a combination of:
a) a therapeutically effective amount of at least one innate immunity modifier,
b) a therapeutically effective amount of at least one immune checkpoint inhibitor, and
c) a therapeutically effective amount of at least one T-cell stimulator.

### Embodiment 23

A pharmaceutical combination for the treatment of cancer comprising a combination of:
a) a first pharmaceutical composition comprising a therapeutically effective amount of at least one innate immunity modifier,
b) a second pharmaceutical composition comprising a therapeutically effective amount of at least one immune checkpoint inhibitor, and
c) a third pharmaceutical composition comprising a therapeutically effective amount of at least one T-cell stimulator.

### Embodiment 24

A pharmaceutical combination for the treatment of cancer comprising a combination of:
a) a therapeutically effective amount of at least one innate immunity modifier which is a selective dipeptidyl peptidase inhibitor;
b) a therapeutically effective amount of at least one immune checkpoint inhibitor selected from a PD-1 axis antagonist or CTLA4 antagonist; and
c) a therapeutically effective amount of at least one T-cell stimulator which is a PEGylated IL-2.

### Embodiment 25

A pharmaceutical combination for the treatment of cancer comprising a combination of:
a) a therapeutically effective amount of at least one innate immunity modifier that is talabostat or a pharmaceutically acceptable salt thereof;
b) a therapeutically effective amount of at least one PD-1 axis antagonist selected from an anti-PD-1 antibody, an anti-PD-L1 antibody, and an anti-PD-2 antibody; and
c) a therapeutically effective amount of at least one an IL2RD selective agonist, optionally multi(2,7-(bis-methoxyPEG-carboxyamide)(9H-fluorene-9-yl)methyl N-carbamate)interleukin-2.

### Embodiment 26

A pharmaceutical combination for the treatment of cancer comprising a combination of:
a) a therapeutically effective amount of at least one innate immunity modifier that is talabostat or a pharmaceutically acceptable salt thereof;
b) a therapeutically effective amount of at least one immune checkpoint inhibitor selected from nivolumab and pembrolizumab; and
c) a therapeutically effective amount of at least IL2RD selective agonist that is multi(2,7-(bis-methoxyPEG-carboxyamide)(9H-fluorene-9-yl)methyl N-carbamate)interleukin-2.

### Embodiment 27

A combination for the treatment of cancer comprises talabostat or a pharmaceutically acceptable salt thereof, nivolumab and multi(2,7-(bis-methoxyPEG-carboxyamide)(9H-fluorene-9-yl)methyl N-carbamate)interleukin-2.

### Embodiment 28

A combination for the treatment of cancer comprises talabostat or a pharmaceutically acceptable salt thereof, pembrolizumab and multi(2,7-(bis-methoxyPEG-carboxyamide)(9H-fluorene-9-yl)methyl N-carbamate)interleukin-2, which comprises RSLAIL-2.

### Embodiment 29

A triple combination for the treatment of cancer consisting of talabostat or a pharmaceutically acceptable salt thereof, nivolumab and multi(2,7-(bis-methoxyPEG-carboxyamide)(9H-fluorene-9-yl)methyl N-carbamate)interleukin-2, which comprises RSLAIL-2.

### Embodiment 30

A triple combination for the treatment of cancer consisting of talabostat or a pharmaceutically acceptable salt thereof, pembrolizumab and multi(2,7-(bis-methoxyPEG-carboxyamide)(9H-fluorene-9-yl)methyl N-carbamate)interleukin-2, which comprises RSLAIL-2.

### Embodiment 31

A triple combination for the treatment of cancer consisting of talabostat mesylate, nivolumab and multi(2,7-(bis-methoxyPEG-carboxyamide)(9H-fluorene-9-yl)methyl N-carbamate)interleukin-2, which comprises RSLAIL-2.

### Embodiment 32

A triple combination for the treatment of cancer consists of talabostat mesylate, pembrolizumab and multi(2,7-(bis-methoxyPEG-carboxyamide)(9H-fluorene-9-yl)methyl N-carbamate)interleukin-2, which comprises RSLAIL-2.

### Embodiment 33

A pharmaceutical composition comprising a combination of: (a) a therapeutically effective amount of at least one innate immunity modifier, (b) a therapeutic effective amount of at least one immune checkpoint inhibitor, and (c) a therapeutically effective amount of at least one T-cell stimulator.

### Embodiment 34

A combination or composition according to any preceding Embodiment further comprising at least one pharmaceutically acceptable excipient and/or carrier.

### Embodiment 35

A composition, combination or method according to any preceding embodiment comprising a T-cell stimulator in a dose range of from about 0.001 mg/kg to about 10 mg/kg; about 0.001 mg/kg to about 5 mg/kg, about 0.001 mg/kg to about 4 mg/kg, about 0.001 mg/kg to about 3 mg/kg, about 0.001 mg/kg to about 2 mg/kg, about 0.001 mg/kg to about 1 mg/kg, about 0.001 mg/kg to about 0.1 mg/kg, about 0.001 mg/kg to about 0.01 mg/kg

### Embodiment 36

A composition, combination or method according to any preceding embodiment comprising an innate immunity modifier in a dose range of from about 0.001 mg/kg to 2 mg/kg, about 0.001 mg/kg to 1 mg/kg, preferably 0.001 mg/kg to 0.5 mg/kg, more preferably about 0.001 mg/kg to 0.2 mg/kg.

### Embodiment 37

A composition, combination or method according to any preceding embodiment comprising an immune checkpoint inhibitor in a dose range of from about 0.1 mg/kg to about 10 mg/kg; from about 1 mg/kg to about 9 mg/kg; from about 1 mg/kg to about 8 mg/kg; from about 1 mg/kg to about 7 mg/kg; from about 1 mg/kg to about 6 mg/kg; from about 1 mg/kg to about 5 mg/kg; from about 1 mg/kg to about 4 mg/kg; from about 1 mg/kg to about 3 mg/kg; from about 1 mg/kg to about 2 mg/kg; from about 1 mg/kg to about 1.5 mg/kg.

### Embodiment 36

A composition, combination or method according to any preceding embodiment comprising a talabostat mesylate in a dose range of from 001 mg/kg to 0.0.024 mg/kg mg/kg, preferably 0.001 mg/kg to 0.017 mg/kg, preferably 0.001 mg/kg to 0.014 mg/kg, more preferably about 0.001 mg/kg to 0.010 mg/kg and more preferably about 0.001 mg/kg to 0.009 mg/kg.

### Embodiment 38

A method of generating a memory anti-tumor immune response in a subject, the method comprising administering to a subject at least one innate immune modifier, at least one immune checkpoint inhibitor, and at least one T-cell stimulator.

All publications, patents, and patent applications disclosed herein are incorporated by reference to the same extent as if each individual publication, patent or patent application was specifically and individually indicated to be incorporated by reference.

### EXAMPLES

### Example 1

### Stimulation of Anti-Tumor Response by Modulating Innate Immunity, T-Cell Response and Check-Point Inhibition in a Mouse Model of Pancreatic Cancer

The anti-tumor efficacy of various combinations of immunomodulatory agents was investigated in a mouse model of pancreatic cancer (Pan 02 syngeneic mouse model).

### Materials and Methods

### Animals:

Six to eight-week-old female C57BL/6 mice were used in the studies as supplied by Beijing Vital River Laboratory Animal Technology Co., Ltd. Mice received food and water ad libitum. The study protocol and the procedures involving the care and use of animals were reviewed and approved by the Institutional Animal Care and Use Committee (IACUC) to ensure compliance with the regulations of the Association for Assessment and Accreditation of Laboratory Animal Care (AAALAC).

### Reagents and Antibodies:

RPMI-1640 medium (Cat. No.: A1049101), Glutamax (Cat. No.: 35050061), Trypsin-EDTA (0.25%) (Cat. No.: 25200-056), Penicillin-Streptomycin (Cat. No.: 15070-063), HBSS (Cat. No.: 14175-095) were procured form Gibco, while Fetal Bovine Serum (FBS) Cat. No.: 004-001-1A was purchased from Biological Industries. The PD-1 antagonist (Cat. No.: BP0146, a mouse anti-PD-1 antibody) was supplied by Crownbio at 6.61 mg/ml. Stock solutions of the PD-1 antagonist at 1 mg/ml concentrations were prepared and kept at 4° C. prior to use. Dosing solutions of the PD-1 antagonist were prepared freshly at a concentration of 1 mg/ml before every administration in sterile phosphate buffered saline (PBS), adjusted to pH 7.0, and administered at a dose of 10 mg/kg intraperitoneally (i.p) per 20 g mouse. (2,7-(bis-methoxyPEG_{10kD}-carboxyamide)(9H-fluorene-9-yl)methyl N-carbamate)_{6avg}interleukin-2, a CD-122 biased cytokine agonist in which recombinant human interleukin-2 (de-1-alanine, 125-serine), is N-substituted with an average of six [(2,7-bis{[methylpoly(oxyethylene)_{10kD}]carbamoyl}-9H-fluoren-9-yl)methoxy]carbonyl moieties at its amino residues (CAS No. 1939126-74-5) was provided by Nektar Therapeutics, referred to as RSLAIL-2 in the accompanying figures and tables, and prepared freshly at a working concentration of 0.08 mg/ml dosing solution, maintained at 4° C., and administered intravenously at a total dose of 0.8 mg/kg. Talabostat mesylate was obtained from a commercial source, and prepared freshly at a working concentration of 0.1 mg/ml before every administration in sterile phosphate buffered saline (pH 7.0), maintained at 4° C., and administered perorally (p.o.) at a total dose of 20 μg per 20 g mouse.

### Tumor Model:

Pan02 tumor cells were maintained in vitro as amonolayer culture in RPMI-1640 medium supplemented with 10% fetal bovine serum at 37° C. in anatmosphere of 5% CO₂ in air. The tumor cells were routinely sub-cultured twice per week bytrypsin-EDTA treatment. The cells in anexponential growth phase were harvested and counted for tumor inoculation. Each mouse was inoculated subcutaneously at the front right flank region with Pan02 tumor cells (3×10⁶) in 0.1 ml of PBS for tumor development. The date of tumor cell inoculation was denoted as Day 0. Five days post tumor implant, mice were sorted into groups of 12 mice with a mean tumor volume of ˜140 mm³ and the test articles and antibody were administered according to the dosing schedules described in Table 1A below:

TABLE 1A
Treatment groups and dosing schedule

| | | | | | | | Day(s) of |
|---|---|---|---|---|---|---|---|
| | | | | | | | Dosing |
| | | | | | | | from the |
| | | | | | | | day of |
| | | | | | | | tumor |
| | | | | | Dosing | | inoculation |
| Group | N* | Treatment | Dose | | Route | Schedule | (Day 0) |
| 1 | 12 | RSLAIL-2 vehicle | 0 | | i.v. | Q9d | Days 5, 14 |
| | | | | | | | and 23 |
| | | Talabostat mesylate | | | p.o. | Qd | from Day 5 |
| | | vehicle | | | | | Day 28 |
| | | | | | | | once a day |
| | | Anti-PD-1 vehicle | | | i.p. | BIW | Days 5, 8, |
| | | | | | | | 12, 15, 19, |
| | | | | | | | 22 and 27 |
| 2 | 12 | Talabostat mesylate | 20 | μg/dose | p.o. | Qd | from Day 5 |
| | | | | | | | to Day 28 |
| | | | | | | | once a day |
| 3 | 12 | RSLAIL-2 | 0.8 | mg/kg | i.v. | Q9D | Days 5, 14 |
| | | | | | | | and 23 |
| 4 | 12 | Anti-PD-1 antibody | 10 | mg/kg | i.p. | BIW | Days 5, 8, |
| | | | | | | | 12, 15, 19, |
| | | | | | | | 22 and 27 |
| 5 | 12 | Talabostat mesylate | 20 | μg/dose | p.o. | Qd | from Day 5 |
| | | | | | | | to Day 28 |
| | | | | | | | once a day |
| | | RSLAIL-2 | 0.8 | mg/kg | i.v. | Q9d | Days 5, 14 |
| | | | | | | | and 23 |
| 6 | 12 | Talabostat mesylate | 20 | μg/dose | p.o. | Qd | from Day 5 |
| | | | | | | | to Day 28 |
| | | | | | | | once a day |
| | | | | | | | Days 5, 8, |
| | | Anti-PD-1 antibody | 10 | mg/kg | i.p. | BIW | 12, 15, 19, |
| | | | | | | | 22 and 27 |
| 7 | 12 | RSLAIL-2 | 0.8 | mg/kg | i.v. | Q9d | Days 8, 17, |
| | | | | | | | 25 |
| | | Anti-PD-1 antibody | 10 | mg/kg | i.p. | BIW | Days 5, 8, |
| | | | | | | | 12, 15, 19, |
| | | | | | | | 22 and 27 |
| 8 | 12 | RSLAIL-2 | 0.8 | mg/kg | i.v. | Q9d | Days 8, 17, |
| | | | | | | | and 25 |
| | | Talabostat mesylate | 20 | μg/dose | p.o. | Qd | from Day 5 |
| | | | | | | | to Day 28 |
| | | | | | | | once a day |
| | | Anti-PD-1 antibody | 10 | mg/kg | i.p. | BIW | Days 5, 8, |
| | | | | | | | 12, 15, 19, |
| | | | | | | | 22 and 27 |

KEY: Q9d = administered on the 9^{th} day, BIW = twice a week, Qd = once daily.
N*—Of the 12 mice, in each group, three from each were sacrificed after three days of the first dose (Day 8) of treatment (IHC—see example 3).
Day 0 is the day of tumor inoculation and days were calculated from Day of first tumor inoculation

The dosing of the agents was started on day 5 after tumor inoculation and continued until Day 28 after tumor inoculation.

Body weight (in grams), and tumor volumes (in mm³) were measured on Days 5, 8, 12, 15, 19, 22, 26 and 29. Tumor volumes were measured twice per week in two dimensions using a caliper and are expressed in mm³ using the formula: V=0.5 a×b² where a and b are the length and width of the tumor, respectively. All procedures, including dosing and tumor and body weight measurement, were conducted in a Laminar Flow Cabinet. Tumor volume, expressed in mm³, was measured with a calliper.

### Results:

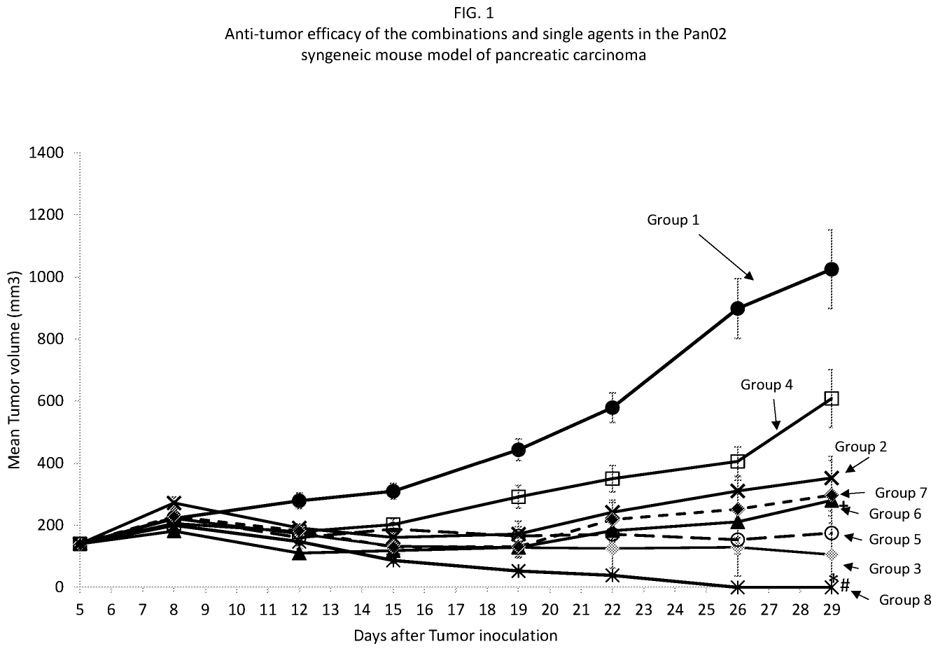

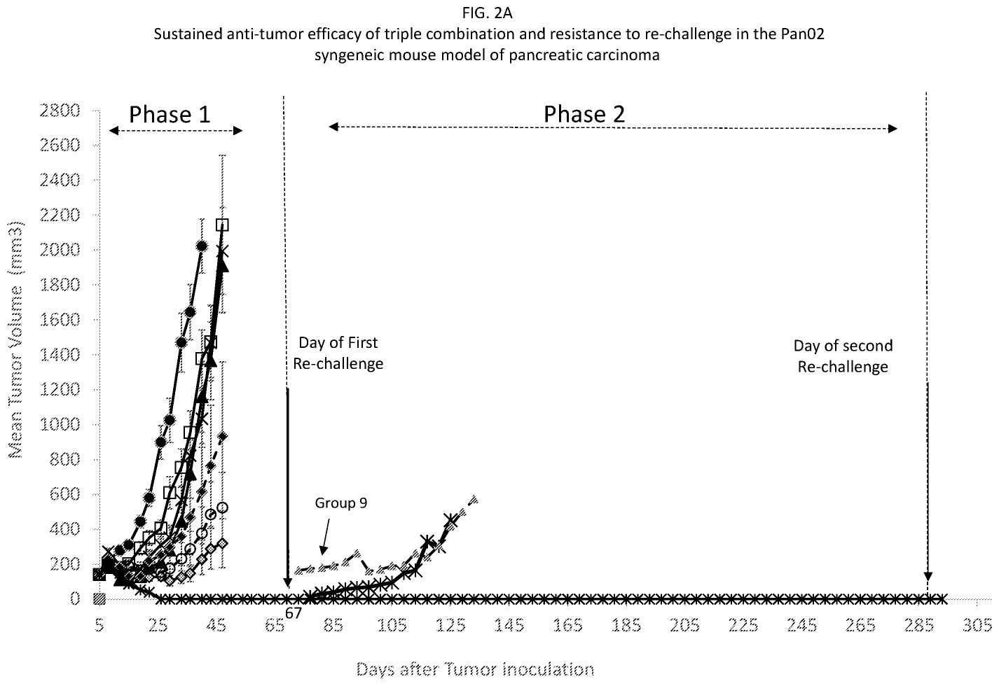

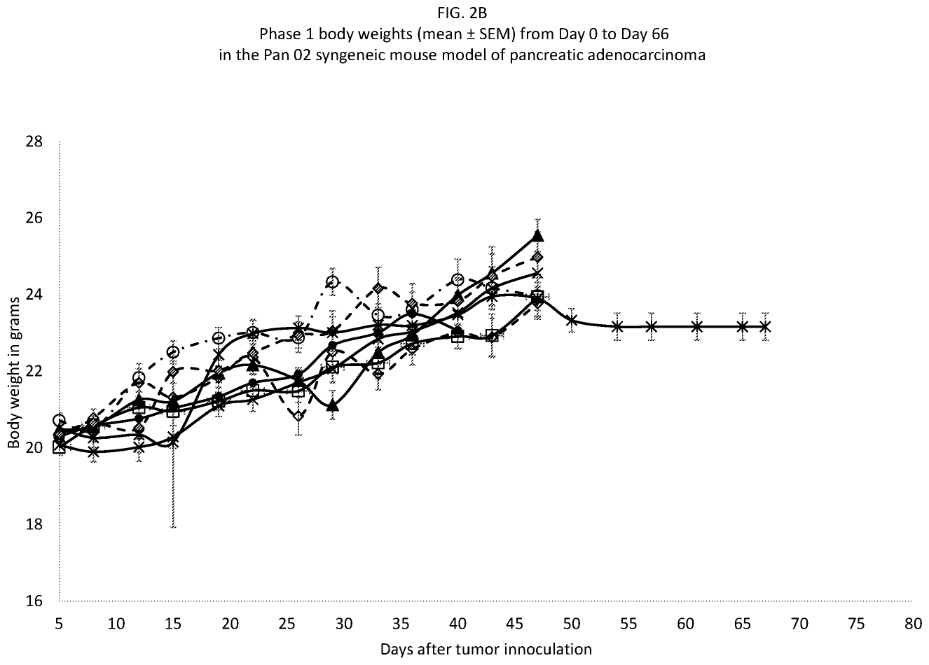

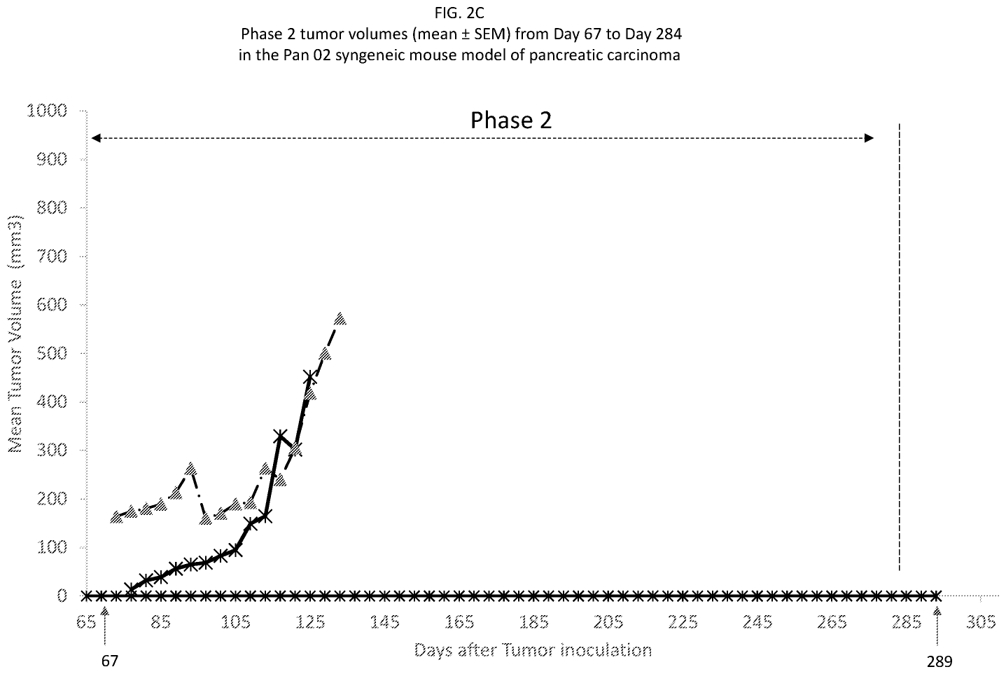

Mice treated with the triple combination of talabostat mesylate (20 μg; Qd), (2,7-(bis-methoxyPEG_{10kD}-carboxyamide)(9H-fluorene-9-yl)methyl N-carbamate)_{6avg}interleukin-2 (0.8 mg/kg; Q9d), and an anti-PD-1 antibody as the PD-1 antagonist (10 mg/kg; BW), Group 8, exhibited remarkable tumor reduction, observed from Day 19 onwards and by Day 29 it was noted that Group 8, exhibited significant tumor reduction when compared with the talabostat mesylate and PD-1 antagonist (group 6), the PD-1 antagonist and RSLAIL-2 (group 7), the talabostat mesylate and RSLAIL-2 (group 5), talabostat mesylate (group 2), RSLAIL-2 (group 3), the PD-1 antagonist (Group 4), and vehicle control (Group 1). See FIGS. 1 and 2A-2B. From Day 26 onwards, the triple combination resulted in complete tumor regression, with 9/9 mice tumor free by Day 26 in contrast to the single agents and the respective doublets. All 9 mice of Group 8 remained tumor free until Day 66, and were then subjected to a first rechallenge on Day 67. The triple combination comprising talabostat mesylate, (2,7-(bis-methoxyPEG_{10kD}-carboxyamide)(9H-fluorene-9-yl)methyl N-carbamate)_{6avg}interleukin-2, and an anti-PD-1 antibody, resulted in complete regression of the tumor.

### Statistical Analysis:

Data related to tumor volume are presented as mean and the standard error of the mean (SEM). Statistical analyses were conducted using Student's t-test. P<0.05 and P<0.001 were considered statistically significant. Percentage tumor reduction was assessed on Days 19, 22, 26 and 29 by using the following formula and as shown in Table 1B below:

% Tumor reduction=(Mean tumor volume^{vehicle control}−Mean tumor volume^{treatmet_group})/Mean tumor volume^{vehicle control}×100Table 1B. Results

| | % Tumor Reduction compared to | | | |
|---|---|---|---|---|
| | vehicle control | | | |
| Groups | Day 19 | Day 22 | Day 26 | Day 29 |
| Talabostat mesylate, 20 micro gram qd, | 61.23 | 58.18 | 65.42 | 65.61 |
| Group 2 | | | | |
| RSLAIL-2, 0.8 mg/kg, q9d, Group 3 | 71.07 | 78.35 | 85.60 | 89.73 |
| PD-1 antagonist, 10 mg/kg, biw, Group 4 | 34.18 | 39.53 | 54.82 | 40.62 |
| Talabostat mesylate + RSLAIL-2, Group 5 | 62.69 | 70.47 | 82.91 | 82.93 |
| Talabostat mesylate + PD-1 antagonist, | 70.91 | 68.46 | 76.52 | 72.65 |
| Group 6 | | | | |
| RSLAIL-2 + PD-1 antagonist, Group 7 | 70.41 | 62.31 | 71.95 | 70.99 |
| Talabostat mesylate + RSLAIL-2 + PD-1 | 88.13 | 93.32 | 100.00 | 100.00 |
| antagonist, Group 8 | | | | |

### Example 2

### Rechallenge Study—Stimulation of Anti-Tumor Memory Response by the Combination of Modulating Innate Immunity, T-Cell Response and Checkpoint Inhibition in a Mouse Model of Pancreatic Cancer

### Material and Methods:

This study is a continuation of the study described in Example 1. 38 days after dosing completion (Day 67 after tumor inoculation), the tumor-free animals exhibiting complete response to combined immunotherapy (Group 8) received a re-challenge of 3×10⁶ Pan02 tumor cells. For the tumor re-challenge study, 6 mice of Group 8 were subcutaneously re-challenged with Pan02 pancreatic adenocarcinoma cells. Tumor volume and body weights were measured twice weekly. Tumor volumes were measured twice per week in two dimensions using a caliper, and the volume was expressed in mm³ using the formula: V=0.5 a×b², where a and b are the length and width of the tumor, respectively. Dosing as well as tumor and body weight measurement were conducted in a Laminar Flow Cabinet.

This rechallenge study was conducted in 2 phases: The initial part of the study (“Phase I”) was as described in Example 1, where 9/9 mice of Group 8 were tumor free by Day 26 and remained tumor free until Day 66. Tumor volumes (in mm³) and body weights (in grams) were measured twice a week and are presented in FIGS. 2B and 2C, respectively. As indicated in FIG. 2B, the body weights of the mice of Group 8, as well as those of other treatment groups did not show any drastic changes, indicating the absence of any toxic effects of the individual agents.

Phase II was initiated on Day 67. A total of 11 mice (5 age appropriate naïve animals and the 6 mice of Group 8 were used and distributed as summarized in Table 2 below.

TABLE 2
Groups for the Phase II
Phase II study design

| Group | N |
|---|---|
| Pan02 tumor-free^{^{#}} animals rechallenged | 6 |
| with Pan02 tumor cells (Group 8) | |
| Age appropriate naïve animals challenged | 5 |
| with Pan02 tumor cells (Group 9) | |

^{^{#}}tumor-free animals refer to Pan02 tumor bearing animals from Phase I that completely responded to the triple combination (talabostat mesylate, PD-1 antagonist and RSLAIL-2)

On Day 67, the mice were injected subcutaneously with Pan02 tumor cells (3×10⁶ tumor cells).

Tumor uptake and tumor growth was observed on the animals from 7 days following inoculation (Day 74). All the age-appropriate naïve animals possessed tumors, with a mean tumor volume of approximately 164±27 mm³ (mean±SEM) and 263±46 mm (mean±SEM) at 7 days and 18 days post challenge, respectively as shown in FIG. 2C. In striking contrast, five of the six mice (83%) of the rechallenged group (Group 8, rechallenged with Pan02 tumor cells) were tumor free at such time points (i.e., they completely rejected the Pan02 tumor rechallenge) and notably remained tumor free until the end of the Phase II (Day 285).

Results: Example 1 demonstrates the synergistic effect of a combination of talabostat mesylate, a PD-1 antagonist and (2,7-(bis-methoxyPEG_{10kD}-carboxyamide)(9H-fluorene-9-yl)methyl N-carbamate)_{6avg}interleukin-2 (RSLAIL-2) in a mouse model of pancreatic cancer. This example illustrates that the foregoing combination is strikingly effective in eliciting anti-tumor immunity, and further demonstrates the efficacy of a therapeutic approach in which an innate arm modifier is combined with an immune checkpoint inhibitor and a T cell stimulator to thereby provide a long term, tumor-specific memory response in treated mice. (FIGS. 2A, 2B, 2C).

### Example 3

### Evaluation of FAP Expression and Immune Infiltrates in Treated Pan02 Tumor Bearing Mice by IHC (Immunohistochemistry)

IHC was performed on tumor samples from animals sacrificed three days after receiving treatment (Day 8) to evaluate FAP expression and presence of immune cell infiltrates. The study was conducted to assess the ability of an exemplary innate immune modifier, talabostat mesylate, to enhance (2,7-(bis-methoxyPEG_{10kD}-carboxyamide)(9H-fluorene-9-yl)methyl N-carbamate)_{6avg}interleukin-2 and PD-1 antagonist efficacy by removing the fibrotic barriers to immune infiltration.

### Materials and Methods:

The tumor samples were taken from the study groups in Table 1. Out of a total of 12 mice, 3 mice from each group were sacrificed on Day 8 after tumor inoculation for the IHC analysis. For evaluation of FAP expression and immune cell infiltrates in the tumor samples, IHC was performed using cryostat sections (8 m thick) of freshly frozen tumor tissues embedded in OCT. The sections were fixed with acetone at −20° C. for 15 minutes and air-dried at room temperature for 15 minutes. Endogenous peroxidases were quenched with 0.3% hydrogen peroxide/PBS washes. Tissue sections were blocked with normal goat serum and then with Avidin and Biotin. Primary antibody or isotype matched controls in 3% (w/v) bovine serum albumin was applied to tissues at concentration of 10 μg/ml at room temperature for 50 minutes. Sections were then incubated with appropriate secondary antibodies, washed, and incubated with diaminobenzidine and counterstained with hematoxylin and staining results were evaluated by our pathologists, who were blinded to the clinical characteristics of the tumor tissues. Antibodies used in the IHC analyses included anti-mouse FAP Ab (ab53066, Abcam), anti-mouse CD8 Ab (14-0808-80, eBiosciences), anti-mouse Ly6G Ab (BE0075-1, Bioxcell) and H&E staining (6765009, ThermoFisher) (as per the manufacturer's protocol).

### Results:

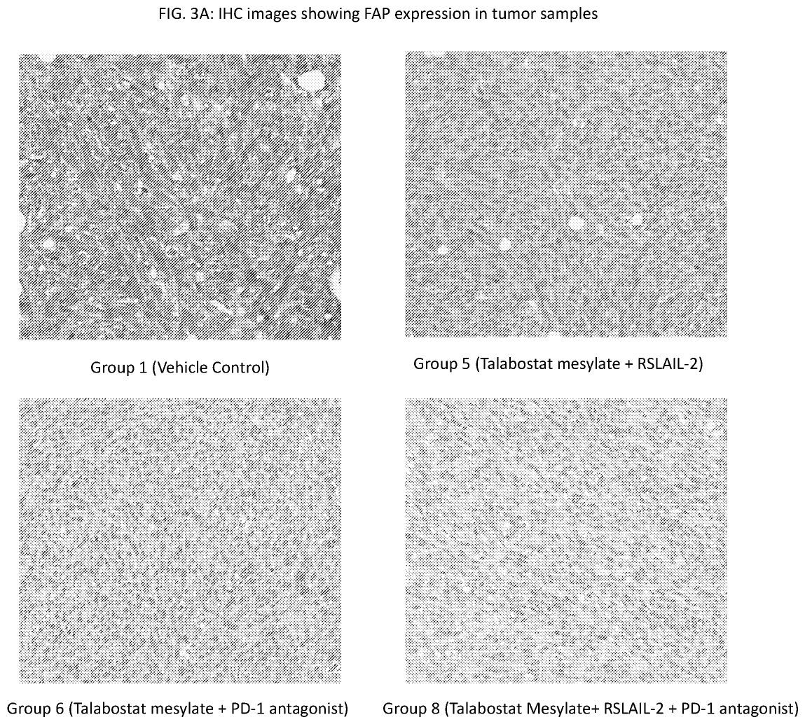

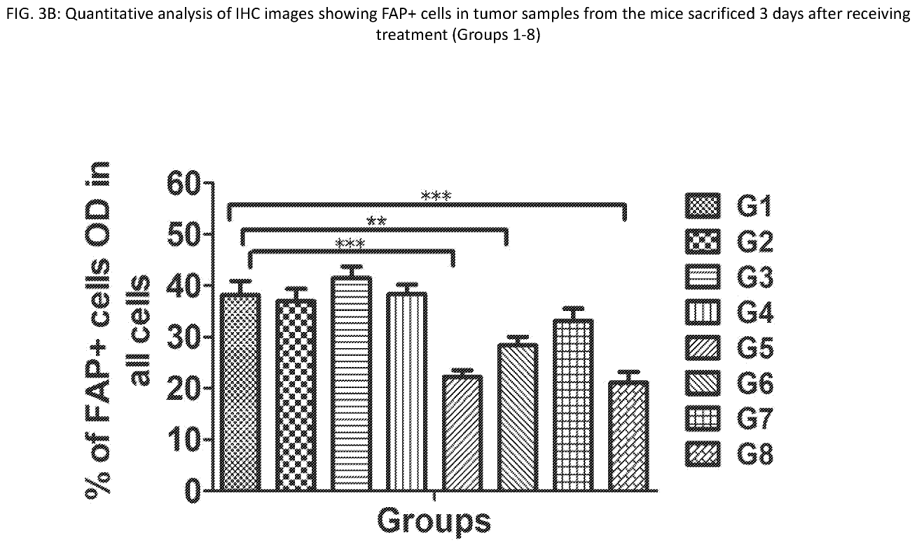

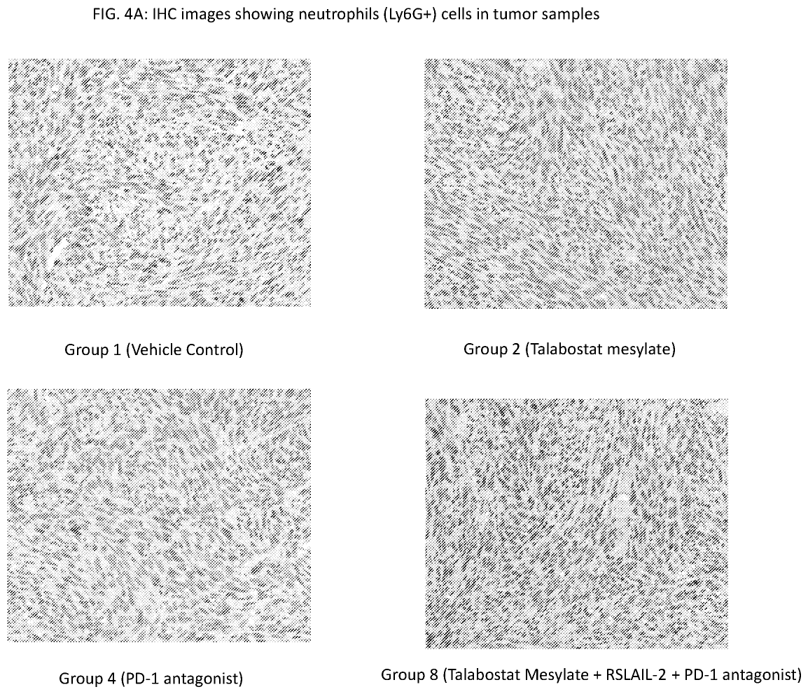

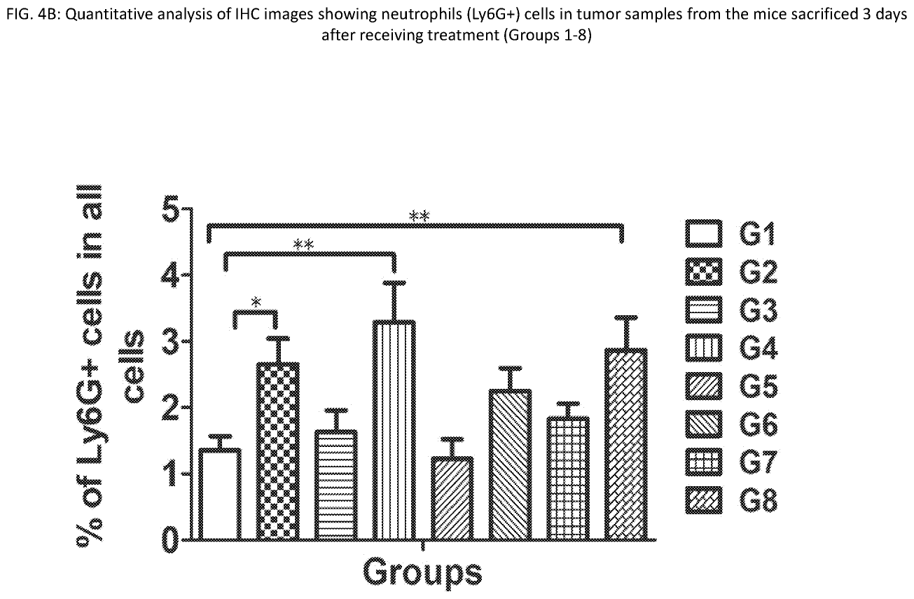

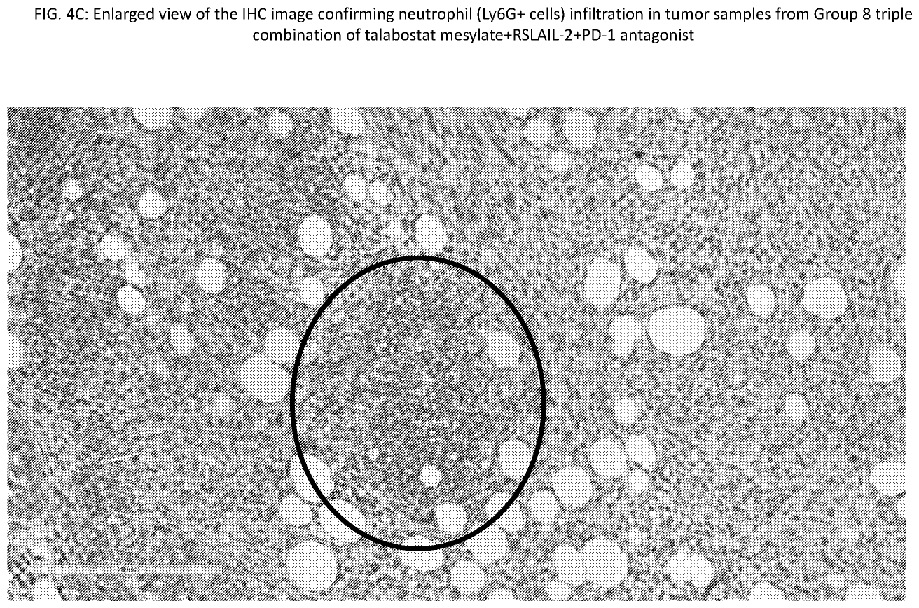

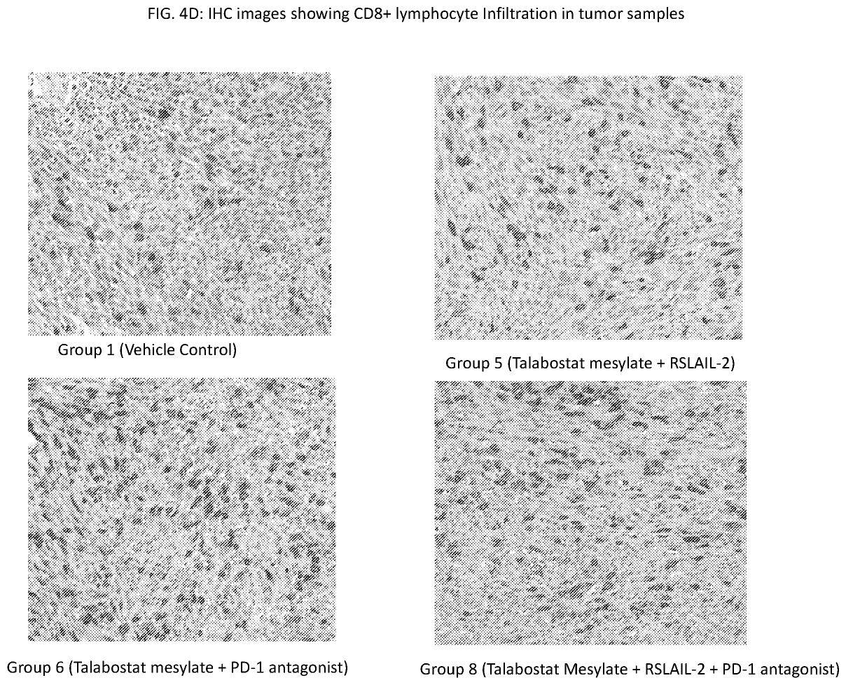

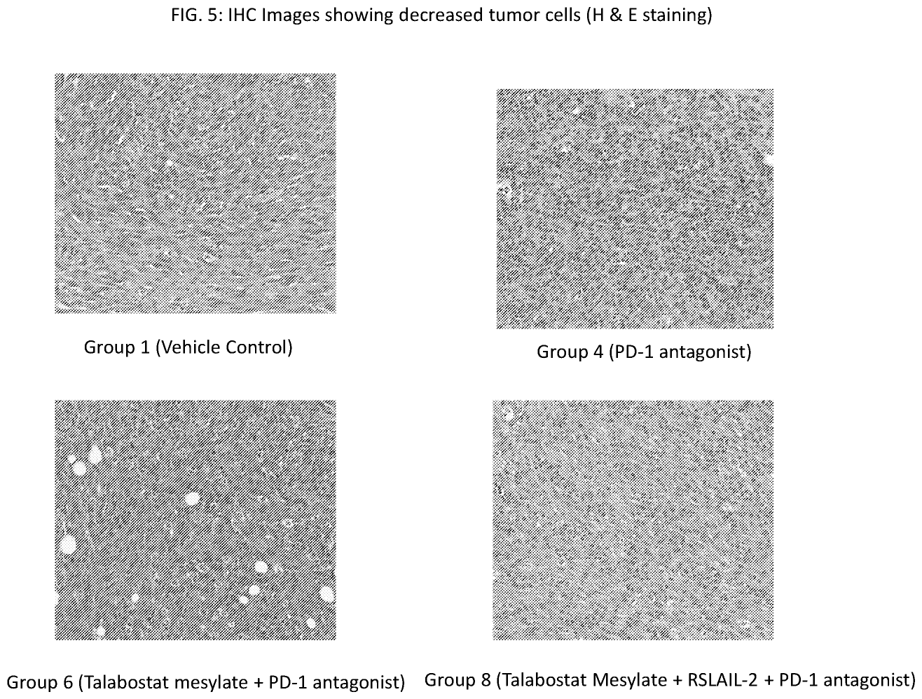

Representative IHC results from tumor samples are shown in panels in FIGS. 3-5. The tumor stroma was easily identified since stromal cells were strongly stained by anti-FAP Ab. FAP was abundantly expressed in the stromal cells. See FIGS. 3A and 3B. FAP was abundantly expressed in >70% of stromal cells in the tumor samples. The groups were compared for FAP (FIGS. 3A, B), for Ly6G staining (FIGS. 4A-4C), for CD8+ T-cell infiltration (FIG. 4D) and tumor cell burden (FIG. 5) through H and E staining.

### FAP Reduction:

IHC of the tumors from satellite animals (sacrificed three days after dosing (Day 8) revealed that talabostat mesylate significantly reduced FAP expression, while the doublet (i.e., two immunotherapeutic agents administered) and triplet (i.e., three immunotherapeutic agents administered) therapies containing talabostat mesylate and (2,7-(bis-methoxyPEG_{10kD}-carboxyamide)(9H-fluorene-9-yl)methyl N-carbamate)_{6avg}interleukin-2 had stronger FAP reduction. The IHC revealed that the talabostat mesylate combination with (2,7-(bis-methoxyPEG_{10kD}-carboxyamide)(9H-fluorene-9-yl)methyl N-carbamate)_{6avg}interleukin-2 and a PD-1 antagonist (triple combination) showed a stronger reduction in FAP as compared to the reduction in FAP observed in tumors treated with a combination of PD-1 antagonist and 2,7-(bis-methoxyPEG_{10kD}-carboxyamide)(9H-fluorene-9-yl)methyl N-carbamate)_{6avg}interleukin-2 (FIGS. 3A, 3B).

### Immune Cell Infiltrate Enhancement:

Tumor samples from the study groups were analyzed for infiltration of immune cells. FIGS. 4A-4C. Mice treated with the triple combination showed a significant increase in Ly6G+ cells (tumoricidal neutrophils), also reflected in tumor samples from mice treated with talabostat mesylate as monotherapy, when compared to the vehicle and PD-1 antagonist alone. The data was analyzed using GraphPad Prism 5. p<0.05 was statistically significant (*, p<0.05; **, p<0.01; ***, p<0.001).

Tumor samples from mice treated with the triple combination also exhibited an increase in CD8+ T-cell infiltration (FIG. 4D), as well as a reduction in H&E staining (FIG. 5) when compared to the other study groups, correlating with an increase in immune response and reduction of tumor burden.

### Example 4

### Evaluation of Cytokine/Chemokine Profiles in Triple Combination-Treated Pan02 Tumor Bearing Mice by Multiplex Cytokine Analysis

Multiplex cytokine analysis was performed on plasma from the mice of Group 8.

### Material and Methods:

Cytokine/chemokine analysis of plasma samples: A group of mice (n=5) was inoculated with Pan02 tumor cells (3×10⁶). The tumor volumes were measured as previously described from day 5 onwards. The immuno-modulatory effect of the triple combination (talabostat mesylate, (2,7-(bis-methoxyPEG_{10kD}-carboxyamide)(9H-fluorene-9-yl)methyl N-carbamate)_{6avg}interleukin-2, and a PD-1 antibody antagonist) was evaluated when the average tumor volume was above 250 mm³ (seen on day 18 after tumor inoculation). 100 μl of blood was collected (pre-treatment), and the mice were administered with the triple combination on Day 18 after tumor inoculation. Seven days following dosing, 100 μl of blood was again collected from the mice (post-treatment). The plasma was separated and stored at −80° C. until analysis. Multiplex serum cytokine/chemokine analysis (using MILLIPLEX® MAP, Merck Millipore) was performed on plasma collected for the pre- and post-treated mice using Luminex analysis; data was normalized.

### Results

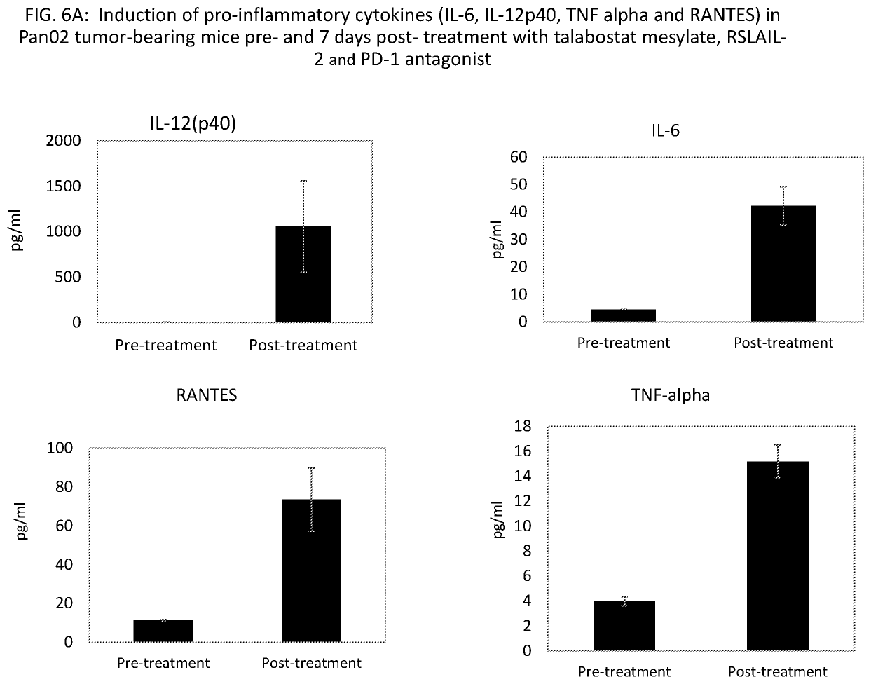

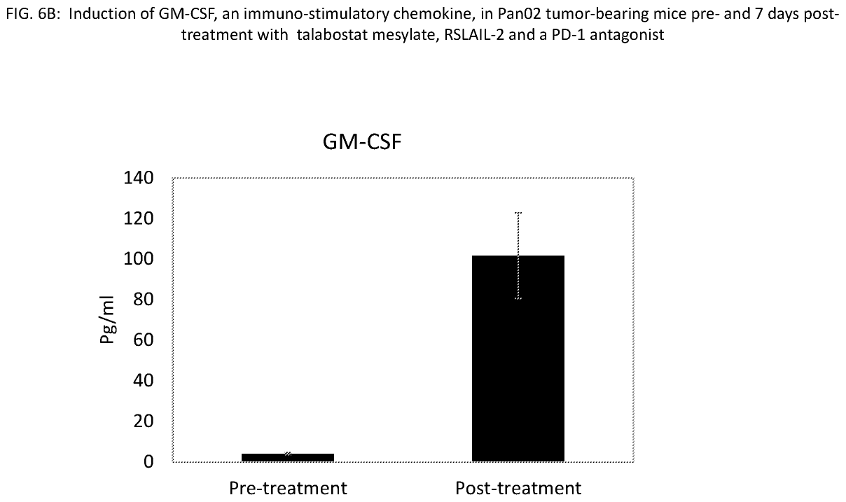

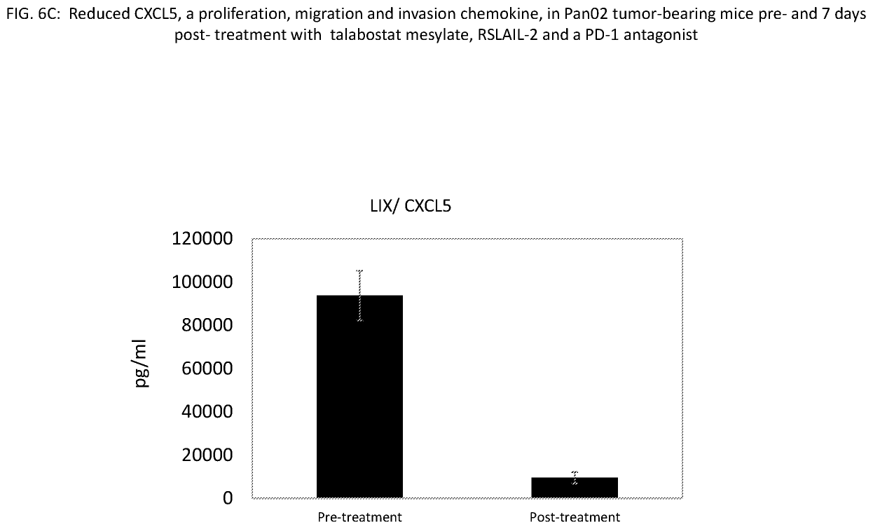

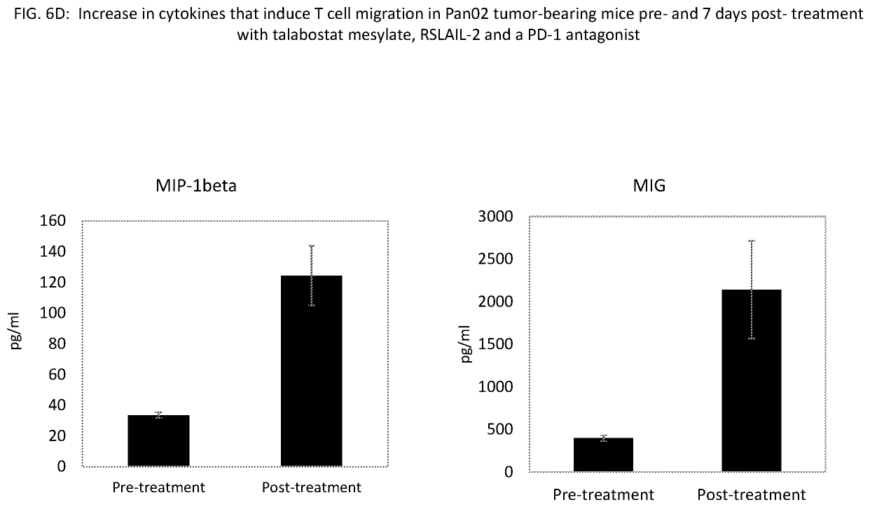

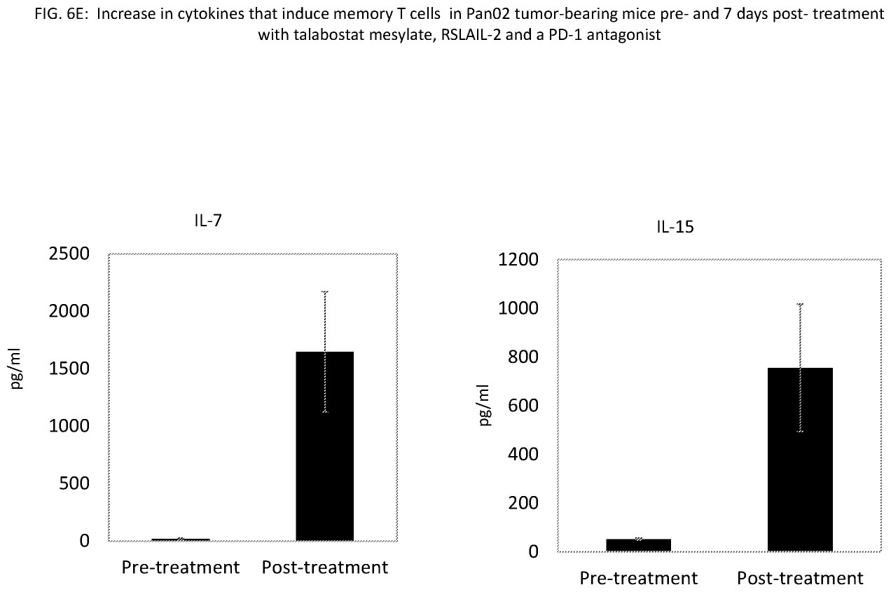

The cytokine/chemokine analysis provided additional confirmation and elucidation of the observed anti-tumor effect in mice treated with the triple combination. Immune-modulation induced by administration of talabostat mesylate, when combined with the PD-1 antagonist and (2,7-(bis-methoxyPEG_{10kD}-carboxyamide)(9H-fluorene-9-yl)methyl N-carbamate)_{6avg}interleukin-2, was observed in the upregulation of pro-inflammatory cytokines including IL-6, IL-12p40, RANTES and TNF alpha (FIG. 6A), as well as in the profiles of chemokines that suppress the immunosuppressive microenvironment including GM-CSF (FIG. 6B) and in cytokines that promote cytotoxic T-cell migration, including MIG and MIP1-beta. (FIG. 6D).

Moreover, the triple combination also showed a synergism in the generation of IL-15 and IL-7, which have the common gamma chain in their receptors. (FIG. 6E). The presence of IL-15 and IL-7 in the immune milieu indicates a reduction in glycolysis which is accompanied by the enhancement of oxidative phosphorylation in activated CD8+ T-cells that skews their phenotype towards memory rather than effector differentiation. The increase in IL-15 and IL-7 (FIG. 6E) indicates that the triple combination may stimulate or enhance a memory T-cell response. Additionally, LIX/CXCL5 (FIG. 6C), which is involved in tumor cell invasion, metastasis and proliferation was decreased after the first dosing with the triple combination. The decrease in LIX suggests that the percentage of the cytotoxic NK cells and M1 macrophages in the tumor will increase and that a decrease in immunosuppressive T-regulatory cells will decrease.

In sum, the illustrative triple combination, when administered to mice in a pancreatic cancer model, was effective to stimulate the innate as well as the adaptive arm of the immune system, thereby resulting in tumor regression. More specifically, the combination of an innate immune modifier, a T-cell stimulator, and an immune checkpoint inhibitor was effective to provide significant immune stimulation as illustrated by:
an increase in pro-inflammatory cytokines (IL-6, IL-12p40, Rantes and TNF alpha);
an increase in immune-stimulatory chemokines (GM-CSF);
an increase in cytokines inducing T-cell migration (MIG, MIP1-beta);
an increase in cytokines associated with memory T-cell response (IL-15 and IL-7);
a decrease in cytokines involved in cell proliferation, invasion and migration (LIX/CXCL5), in plasma (or blood) when compared to a sample of plasma (or blood) taken prior to treatment.

### Example 5

Evaluation of Memory Effector T Cells in Triple Combination Treated Pan02 Tumor Bearing Mice after Re-Re-Challenge by Flow Cytometry (FACS)

The development of anti-tumor immunity as measured by effector memory CD8+ T cell generation was explored in a Pan02 mouse model as described in Examples 1 and 2. Material and methods

Mice that, upon treatment with the triple combination therapy (talabostat mesylate+PD1 antagonist+RSLAIL-2), became tumor-free and showed no tumor growth upon tumor re-challenge in a Pan02 mouse model of pancreatic adenocarcoinoma (Example 2), were again re-challeneged on Day 285 (from the day of first tumor inoculation) by inoculation with Pan02 tumor cells (3×10⁶). Group A=re-challenged mice (n=5). In parallel as a control, naïve mice (n=3, Group B) were inoculated with the same number of Pan02 tumor cells, while naïve mice (n=2, Group C) received no tumor cell inoculum. These mice were then sacrificed 4 days following re-inoculation (Day 289), and the spleens were harvested. Single cell suspensions were prepared, and the splenocytes were stained with anti-CD8 PerCP (cat no. 561798, clone no. 17A2, Biolegend) and anti-CD3 FITC (cat no. 100734, clone no. 53-6.7, Biolegend). Further staining was also performed for CD44 and CD62L with PE-labeled anti-CD44 (cat no. 103024, clone no. IM7, Biolegend) and APC labelled anti-CD62L (cat no. 104412, clone no. MEL-14, Biolegend) respectively. The splenocytes were fixed and subjected to flow cytometric analysis on FACS LRSfortessa (BD Biosciences, San Jose, Calif.) and quantified using by Kaluza software (Beckman Coulter). CD8+ effector memory cells are defined as CD62L-lo/CD44hi.

### Statistical Analysis

The Bartlett test was used to test homogeneity of variance and normality. If the p-value of Bartlett test was no less than 0.05, ANOVA and two sample t-test were used to compare group means. If the p-value of Bartlett test was less than 0.05, Kruskal-wallis test and Wilcoxon rank sum test were used to compare group means.

### Results:

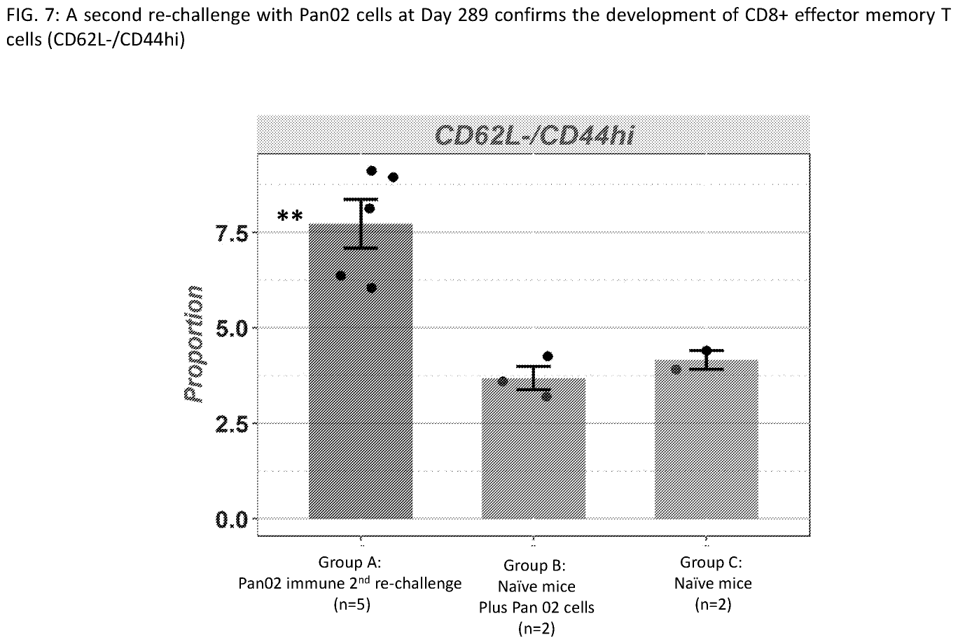

FACs analysis showed the development of CD62L-veCD44hi effector memory cells (CD8+ cells). The effector cell numbers were significantly higher in the re-rechallenged group (Group A), when compared to the naïve controls (Groups B and C). These data confirm the generation of a CD8+ effector memory T cell response in mice that have developed immunity to Pan02 tumor cells resulting from the triple combination therapy (i.e., talabostat mesylate+PD1 antagonist+RSLAIL-2 (FIG. 7)).

### Example 6

### Evaluation of Anti-Tumor Efficacy and Anti-Tumor Memory in a Mouse WEHI-164 Sarcoma Model

The aim of this study was to investigate the anti-tumor effect and degree of anti-tumor immunity resulting from administration of a combination of immunomodulators (i.e., an exemplary triple combination of talabostat mesylate, a PD-1 antagonist, and RSLAIL-2) in a mouse model of sarcoma.

### Material and Methods:

Animals: Six to eight-week-old female C57BL/6 mice were used in the studies as supplied by Beijing Vital River Laboratory Animal Technology Co., Ltd. Mice received food and water ad libitum. The study protocol, the procedures involving the care and use of animals were reviewed and approved by the Institutional Animal Care and Use Committee (IACUC) to ensure compliance with the regulations of the Association for Assessment and Accreditation of Laboratory Animal Care (AAALAC).

Reagents and Antibodies: RPMI-1640 medium (Cat. No.: A1049101), Glutamax (Cat. No.: 35050061), Trypsin-EDTA (0.25%) (Cat. No.: 25200-056), Penicillin-Streptomycin (Cat. No.: 15070-063), HBSS (Cat. No.: 14175-095) were procured form Gibco, while Fetal Bovine Serum (FBS) Cat. No.: 004-001-1A was purchased from Biological Industries. PD-1 antagonist (anti-PD-1 antibody; Cat. No.: BP0146 procured from BioXcell) was supplied by Crown Bioscience, Inc. at 6.61 mg/ml. Stock solutions of PD iantagonist, at 1 mg/ml concentrations were prepared and kept at 4° C. prior to use. Dosing solutions of PD-1 antagonist were freshly prepared at a concentration of 1 mg/ml, before every administration in sterile phosphate buffered saline (PBS), pH 7.0 and administered a dose of 10 mg/kg, intraperitoneally (i.p) per 20 g mouse. Talabostat mesylate was acquired from a commercial source and freshly prepared at a working concentration of 0.1 mg/ml before every administration in sterile phosphate buffered saline (pH 7.0), maintained at 4° C., and administered perorally (p.o) a total dose of 20 μg per 20 g mouse. RSLAIL-2 was provided by Nektar and freshly prepared at a working concentration of 0.08 mg/ml, maintained at 4° C., and administered intravenously (i.v.) a dose of 0.8 mg/kg per 20 g mouse.

Tumor Model: The WEHI 164 tumor cells were maintained in vitro as a monolayer culture in RPMI-1640 medium supplemented with 10% fetal bovine serum at 37° C. in an atmosphere of 5% CO₂ in air. The tumor cells were routinely sub-cultured twice per week by trypsin-EDTA treatment. The cells in an exponential growth phase were harvested and counted for tumor inoculation.

Each mouse was inoculated subcutaneously at the front right flank region with the respective tumor cells (1×10⁶) in 0.1 ml of PBS for tumor development. The date of tumor cell inoculation was denoted as day 0. Five days (day 5) post tumor implant, mice were sorted into a group of 6 mice with a mean tumor volume of 110 mm³. Mice were subjected to dosing schedule as below, for only the triple combination (table 3):

TABLE 3
Dosing schedule

| Test article | Dose | | Route | frequency |
|---|---|---|---|---|
| RSLAIL-2 | 0.8 | mg/kg | i.v. | Q9d |
| Talabostat mesylate | 20 | μg/dose | p.o. | Qd |
| PD-1 antagonist | 10 | mg/kg | i.p. | BIW |

KEY: Q9d = administered on the 9^{th} day, BIW = twice a week, Qd = once daily.

Dosing of the test articles was initiated on Day 5 following initial tumor inoculation and continued until Day 35 following tumor inoculation. Tumor volumes were measured on Day 7, Day 11, Day 14, Day 17, Day 20, Day 24, Day 27, Day 31 and Day 34.

Tumor size and body weights were measured twice weekly. Tumor volumes were measured twice per week in two dimensions using a caliper, and the volumes were expressed in mm³ using the formula: V=0.5 a×b² where a and b are the length and width of the tumor, respectively. Dosing as well as tumor and body weight measurements were conducted in a Laminar Flow Cabinet.

Rechallenge: 102 days after the cessation of treatment (i.e., Day 137 after tumor inoculation), animals (3 out of 6) remained tumor-free, exhibiting complete response. These animals received a re-challenge of 1×10⁶ WEHI 164 cells on Day 137, and the mice were monitored up to and beyond Day 150. A set of three C57BL/6 naïve mice were inoculated simultaneously with 1×10⁶ WEHI 164, as control.

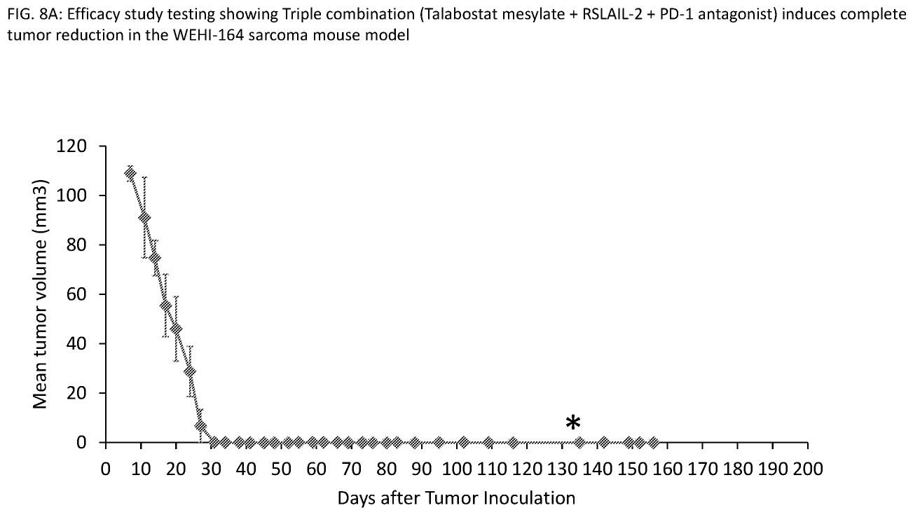

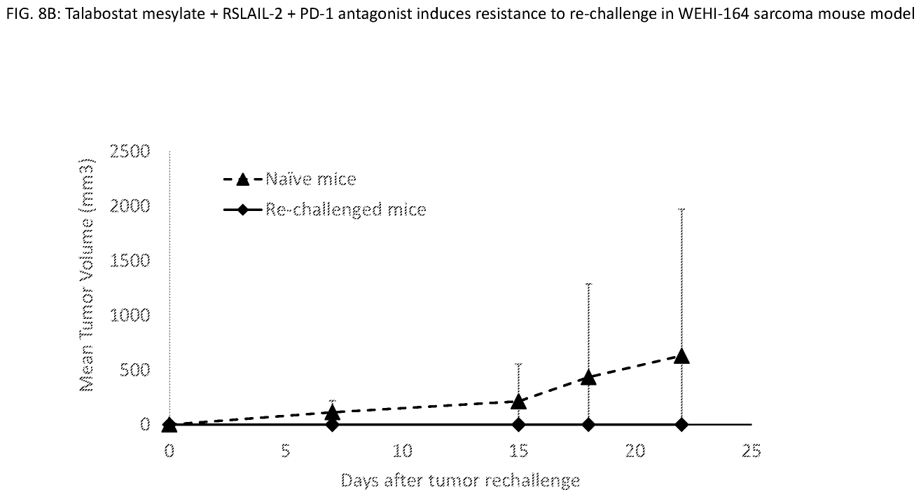

Results: Treatment of established tumors (˜110 mm³) with the exemplary triple combination resulted in complete tumor regression in 3 of the 6 mice by day 35 (50% tumor free). These 3 mice remained tumor free until 137 days from the day tumor inoculation. On Day 137, the 3 mice were re-challenged with 1×10⁶ WEHI 164, and all mice remained tumor free until and beyond Day 150 (100% tumor free); this was in contrast to the naïve mice. This data demonstrates the generation of long-term tumor-specific memory response (FIGS. 8A and 8B).

### Example 7

### Evaluation of Anti-tumor Efficacy and Anti-Tumor Memory in a Mouse MC-38 Colon Cancer Model

### Material and Methods:

Animals: Six to eight-week-old female C57BL/6 mice were used in the studies as supplied by Beijing Vital River Laboratory Animal Technology Co., Ltd. Mice received food and water ad libitum. The study protocol, the procedures involving the care and use of animals were reviewed and approved by the Institutional Animal Care and Use Committee (IACUC) to ensure compliance with the regulations of the Association for Assessment and Accreditation of Laboratory Animal Care (AAALAC).

Reagents and Antibodies: RPMI-1640 medium (Cat. No.: A1049101), Glutamax (Cat. No.: 35050061), Trypsin-EDTA (0.25%) (Cat. No.: 25200-056), Penicillin-Streptomycin (Cat. No.: 15070-063), HBSS (Cat. No.: 14175-095) were procured form Gibco, while Fetal Bovine Serum (FBS) Cat. No.: 004-001-1A was purchased from Biological Industries. PD1 antagonist (anti-PD1 antibody; Cat. No.: BP0146 procured from BioXcell) was supplied by Crownbio Biosciences, Inc. at 6.61 mg/ml. Stock solutions of PD-1 antagonist, at 1 mg/ml concentrations were prepared and kept at 4° C. prior to use. Dosing solutions of PD1 antagonist were freshly prepared at a concentration of 1 mg/ml, before every administration in sterile phosphate buffered saline (PBS), pH 7.0 and administered a dose of 10 mg/kg, intraperitoneally (i.p) per 20 g mouse. The test article talabostat mesylate, was acquired from a commercial source, and freshly prepared at a working concentration of 0.1 mg/ml before every administration in sterile phosphate buffered saline (pH 7.0), maintained at 4° C., and administered perorally (p.o) a total dose of 20 μg per 20 g mouse. RSLAIL-2 was provided by Nektar Therapeutics and freshly prepared at a working concentration of 0.08 mg/ml, maintained at 4° C., and administered intravenously (i.v.) at a dose of 0.8 mg/kg per 20 g mouse.

Tumor Model: The MC38 colon adenocarcinoma cells were maintained in vitro as a monolayer culture in RPMI-1640 medium supplemented with 10% fetal bovine serum at 37° C. in an atmosphere of 5% CO₂ in air. The tumor cells were routinely sub-cultured twice per week by trypsin-EDTA treatment. The cells in an exponential growth phase were harvested and counted for tumor inoculation.

Each mouse was inoculated subcutaneously at the front right flank region with the respective tumor cells (1×10⁶) in 0.1 ml of PBS for tumor development. The date of tumor cell inoculation was denoted as day 0. Five days post tumor implant, mice were sorted into group of 6 mice with a mean tumor volume of ˜120 mm³. Mice were subjected to the dosing schedule as below for components of the triple combination (Table 4):

TABLE 4
Dosing schedule

| Treatment | Dose | | Route | Frequency |
|---|---|---|---|---|
| RSLAIL-2 | 0.8 | mg/kg | i.v. | Q9d |
| Talabostat mesylate | 20 | μg/dose | p.o. | Qd |
| PD-1 antagonist | 10 | mg/kg | i.p. | BIW |

KEY: Q9d = administered on the 9^{th} day, BIW = twice a week, Qd = once daily.

Dosing of the agents was started on Day 5 following tumor inoculation and continued until Day 35 following tumor inoculation. Tumor volumes were measured on Day 10, Day 13, Day 16, Day 18, Day 21, Day 25, Day 28, Day 32 and Day 35.

Tumor volumes were measured in two dimensions using a caliper, and the volumes were expressed in mm³ using the formula: V=0.5 a×b² where a and b are the length and width of the tumor, respectively. Dosing and tumor and body weight measurements were conducted in a Laminar Flow Cabinet.

Re-challenge: 101 days after the cessation of treatment (i.e. Day 136 following tumor inoculation), all animals remained tumor-free (i.e., exhibiting a complete response). The animals received a re-challenge of 1×10⁶ MC38 cells on Day 136 and were then monitored to Day 150 and beyond. A group of three C57BL/6 naïve mice was inoculated simultaneously with 1×10⁶ MC-38 as control.

### Results:

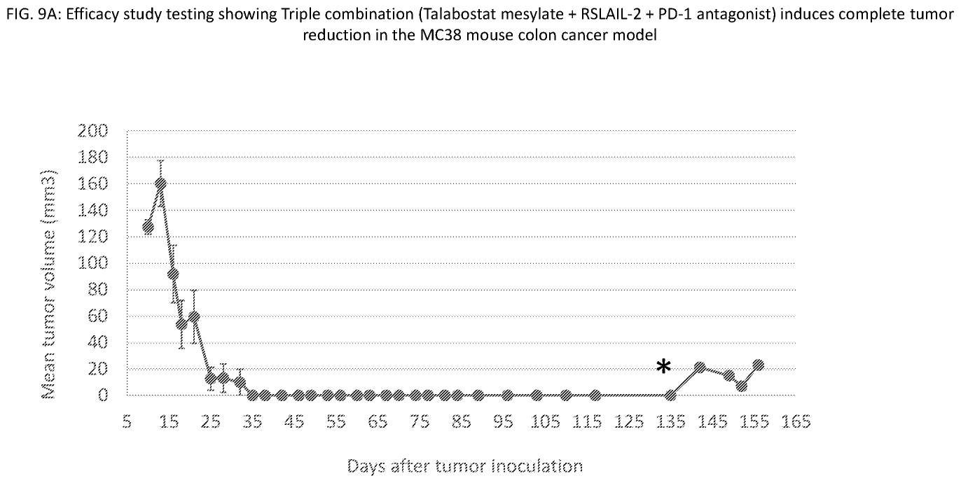

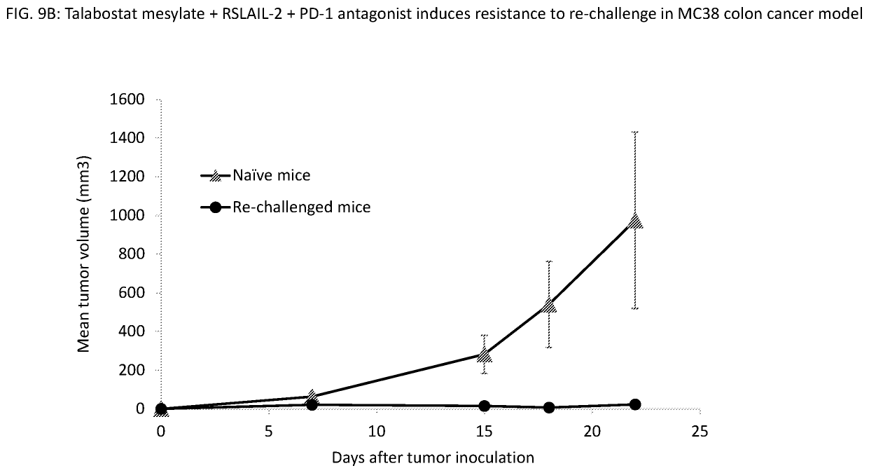

The treatment of established tumors (˜120 mm³) with the triple combination resulted in 100% tumor-free mice (6/6) in the MC38 model by Dday 35. These 6 mice, however, remained tumor free until Day 136 from the day of initial tumor inoculation (FIG. 9a). On Day 136, 6 mice were rechallenged with 1×10⁶ MC-38 cells. Of this group, 6/6 re-challenged mice (only 1 mouse showed a slight increase in tumor volume) rejected tumor growth unlike the naïve mice, demonstrating the generation of a long term tumor-specific memory response in the MC38 mouse model (FIG. 9B).

Various syngeneic mouse models were evaluated and it was discovered that the tumor models that were responsive to the triple combination had high densities of tumor-associated macrophages, while those models that were less responsive had low macrophage densities (data not shown). Thus, it appears that talabostat mesylate-stimulated macrophages may rapidly prime the tumor microenvironment for other immune effector cells, those of which are similarly primed by a combination of checkpoint inhibition and interleukin stimulation.

What is claimed is:

1. A method of treating a subject having cancer, the method comprising administering to the subject an innate immune modifier, an immune checkpoint inhibitor, and a T-cell stimulator.

2. The method of claim 1, wherein the innate immune modifier is a selective dipeptidyl peptidase inhibitor.

3. The method of claim 2, wherein said selective dipeptidyl peptidase inhibitor is selected from the group consisting of talabostat, its analogs, prodrugs, and stereoisomers; and pharmaceutically acceptable salts, hydrates and solvents of any of the foregoing.

4. The method of claim 3, wherein said selective dipeptidyl peptidase inhibitor is talabostat or a pharmaceutically acceptable salt thereof.

5. The method of claim 4, wherein the selective dipeptidyl peptidase inhibitor is talabostat mesylate.

6. The method of any one of claims 1-5, wherein the immune checkpoint inhibitor is either a PD-1 axis antagonist or a CTLA-4 antagonist.

7. The method of claim 6, wherein the PD-1 axis antagonist is selected from a PD-1 antagonist, a PD-L1 antagonist, and a PD-L2 antagonist.

8. The method of claim 7, wherein the PD-1 axis antagonist is a PD-1 antagonist selected from the group consisting of ANA011, AUNP-12, BGB-A317, KD033, pembrolizumab, MCLA-134, mDX400, MEDI00680, muDX400, nivolumab, PDR001, PF-06801591, REGN-2810, SHR-1210, STI-A1110, TSR-042, ANB011, 244C8, 388D4, TSR042, BCD100, camrelizumab, JNJ63723283, JS001, spartalizumab, cemiplimab, tislelizumab, XCE853, and combinations thereof.

9. The method of claim 7, wherein the PD-1 axis antagonist is a PD-L1 antagonist selected from the group consisting of avelumab, BMS-936559, CA-170, durvalumab, MCLA-145, SP142, STI-A1011, STI-A1012, STI-A1010, STI-A1014, A110, KY1003, and atezolimumab.

10. The method of claim any one of claims 1-9, wherein the T-cell stimulator is an interleukin-2 receptor beta (IL-2Rβ) selective agonist.

11. The method of claim 10, wherein the interleukin-2 receptor beta selective agonist comprises an interleukin-2 protein conjugated to polyethylene glycol.

12. The method of claim 11, wherein the interleukin-2 receptor beta selective agonist is (2,7-(bis-methoxyPEG_{10kD}-carboxyamide)(9H-fluorene-9-yl)methyl N-carbamate)₆ₐᵥ interleukin-2.

13. The method of claim 1, comprising administering to the subject, talabostat mesylate, a PD-1 axis antagonist, and (2,7-(bis-methoxyPEG_{10kD}-carboxyamide)(9H-fluorene-9-yl)methyl N-carbamate)₆ₐᵥ interleukin-2.

14. The method of claim 13, wherein the talabostat mesylate, the PD-1 axis antagonist, and (2,7-(bis-methoxyPEG_{10kD}-carboxyamide)(9H-fluorene-9-yl)methyl N-carbamate)₆ₐᵥ interleukin-2 are administered together, comprised in a single dosage form.

15. The method of claim 13, wherein the talabostat mesylate, the PD-1 axis antagonist, and (2,7-(bis-methoxyPEG_{10kD}-carboxyamide)(9H-fluorene-9-yl)methyl N-carbamate)₆ₐᵥ interleukin-2 are each administered as separate, individual dosage forms.

16. The method of any one of claims 1-15, wherein the cancer is selected from the group consisting of pancreatic cancer, colorectal cancer, fibrosarcoma, colon cancer, colon adenocarcinoma or sarcoma, non-small cell lung cancer, prostate cancer, hormone refractory prostate cancer, treatment induced neuroendocrine prostate cancer, castration resistant prostate cancer, breast cancer, ovarian cancer, gastric cancer, malignant melanoma, head and neck cancer, liver cancer, small cell lung cancer, thyroid cancers, kidney cancer, cancer of the bile duct, brain cancer, cervical cancer, maxillary sinus cancer, bladder cancer, esophageal cancer, Hodgkin's disease, non-Hodgkin's lymphoma, acute myeloid leukemia and adrenocortical cancer.

17. The method of claim 16, wherein the cancer is pancreatic cancer.

18. A pharmaceutical combination for use in treating a subject having cancer, the combination comprising:
a) a therapeutically effective amount of an innate immunity modifier,
b) a therapeutically effective amount of an immune checkpoint inhibitor, and
c) a therapeutically effective amount of a T-cell stimulator.

19. The pharmaceutical combination of claim 18, wherein (a) the innate immunity modifier is a selective dipeptidyl peptidase inhibitor, (b) the immune checkpoint inhibitor is either a PD-1 axis antagonist or a CTLA-4 antagonist; and (c) the T-cell stimulator comprises an interleukin-2 protein conjugated to polyethylene glycol.

20. The pharmaceutical combination of claim 19, wherein (a) the selective dipeptidyl peptidase inhibitor is talabostat or a pharmaceutically acceptable salt thereof; (b) the immune checkpoint inhibitor is a PD-1 axis antagonist selected from an anti-PD-1 antibody, an anti-PD-L1 antibody, and an anti-PD-2 antibody; and (c) the interleukin-2 protein conjugated to polyethylene glycol is (2,7-(bis-methoxyPEG_{10kD}-carboxyamide)(9H-fluorene-9-yl)methyl N-carbamate)₆ₐᵥ interleukin-2.

21. The pharmaceutical combination of claim 20 comprising (a) a therapeutically effective amount of talabostat or a pharmaceutically acceptable salt thereof, (b) a therapeutically effective amount of nivolumab or pembrolizumab; and (c) a therapeutically effective amount of (2,7-(bis-methoxyPEG_{10kD}-carboxyamide)(9H-fluorene-9-yl)methyl N-carbamate)₆ₐᵥ interleukin-2.

22. The pharmaceutical combination of claim 21, comprising (a) a therapeutically effective amount of talabostat mesylate, (b) a therapeutically effective amount of nivolumab or pembrolizumab, and (c) a therapeutically effective amount of (2,7-(bis-methoxyPEG_{10kD}-carboxyamide)(9H-fluorene-9-yl)methyl N-carbamate)₆ₐᵥ interleukin-2.

23. The pharmaceutical combination of any one of claims 18-22, comprised in a kit.

24. The pharmaceutical combination of any one of claims 18-22, for use in treating a subject having a cancer selected from the group consisting of pancreatic cancer, colorectal cancer, fibrosarcoma, colon cancer, colon adenocarcinoma or sarcoma, non-small cell lung cancer, prostate cancer, hormone refractory prostate cancer, treatment induced neuroendocrine prostate cancer, castration resistant prostate cancer, breast cancer, ovarian cancer, gastric cancer, malignant melanoma, head and neck cancer, liver cancer, small cell lung cancer, thyroid cancers, kidney cancer, cancer of the bile duct, brain cancer, cervical cancer, maxillary sinus cancer, bladder cancer, esophageal cancer, Hodgkin's disease, non-Hodgkin's lymphoma, acute myeloid leukemia and adrenocortical cancer.

25. The pharmaceutical combination of claim 24, for use in treating a subject having pancreatic cancer.

26. The pharmaceutical combination of claim 24 wherein the tumor has a macrophage density of at least 20%, at least 30%, at least 40% or at least 50%.


SEQUENCE LISTING (1 sequence)

SEQ ID NO: 1 - protein, 288 aa, Homo sapiens
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVICSRAARGTIGARRTGQPLKEDPSAVPVFSVDYGELDFQWREKTPEPPVPCVPEQTEYATIVFPSGMGTSSPARRGSADGPRSAQPLRPEDGHCSWPL